(19) United States
(12) Patent Application Publication
(10) Pub. No.: US 2013/0136714 A1
(43) Pub. Date: May 30, 2013

(54) FUNCTIONALIZED POLYMER NANOPARTICLES AND THE PHARMACEUTICAL USE THEREOF

(71) Applicants: Li-Fang Wang, Kaohsiung City (TW); Shih-Jer Huang, NEW TAIPEI CITY (TW); Zhi-Rong Hsu, KAOHSIUNG CITY (TW)

(72) Inventors: Li-Fang Wang, Kaohsiung City (TW); Shih-Jer Huang, NEW TAIPEI CITY (TW); Zhi-Rong Hsu, KAOHSIUNG CITY (TW)

(73) Assignee: KAOHSIUNG MEDICAL UNIVERSITY, Kaohsiung City (TW)

(21) Appl. No.: 13/572,170

(22) Filed: Aug. 10, 2012

(30) Foreign Application Priority Data
Nov. 28, 2011 (TW) 100143627

(51) Int. Cl. C08G 81/02; A61P 35/00; A61K 31/77; A61P 29/00; B82Y 30/00

(52) U.S. Cl. 424/78.3; 525/539; 525/451; 525/421; 525/422; 525/54.1; 977/773

(57) ABSTRACT

PEO-PPO-PEO polymers and vinyl monomers are used to prepare several block copolymers via consecutive atom transfer radical polymerization (ATRP). The block copolymers provide good delivery characteristics and can be used as a gene/drug delivery carrier for therapy and diagnosis.

### CROSS REFERENCE TO RELATED APPLICATIONS

This application claims benefit under 35 U.S.C. §119 of TW Application No. 100143627, filed Nov. 28, 2011, the contents of which are incorporated by reference as if fully set forth.

### FIELD OF THE INVENTION

The present invention relates to a polymer nanoparticle, in particular a polymer nanoparticle containing polyethylene oxide (PEO). Preferably, the polymer nanoparticle functions as a drug carrier or gene carrier and exhibits its pharmaceutical use for treatment and diagnosis.

### BACKGROUND OF THE INVENTION

The development of the recent biopharmaceutics usually uses nanotechnology. This brings drug/gene therapies from the bench top to the bedside. A nanomaterial refers to the material smaller than 100 nm, which can be divided into zero-dimensional nanoparticle, one-dimensional nanowire (or nanotube), two-dimensional nanofilm and three-dimensional nanoblock according to the structural dimensions. Cationic polymers prepared with poly 2-(dimethylamino)ethyl methacrylate (pDMAEMA), polyethylene imine (PEI), polylysine etc. have been used to coat a gene drug. The formed nanoparticle must meet the criteria of no cytotoxicity, hydrophilicity and biocompatibility, and then can be used in a living body. However, most cationic polymers exhibit higher cytotoxicity due to physicochemical factors such as their poor biocompatibility and the permeability blocking of the cell membrane. These are the first issues to be overcome in the current clinical application.

Layman, J. M. et al. used various 2-(dimethylamino)ethyl methacrylate (DMAEMA) to form polyplexes with DNA, and the results show that the increased PDMAEMA blocks increase the gene expression but decrease the biocompatibility (Biomacromolecules 2009, 10 (5), 1244-52). Agarwal, A. et al. used Pluronic® and PDEAEMA to make a block copolymer as a gene delivery carrier (J. Control Release 2005, 103 (1), 245-58). The combination of Pluronic® and PDMAEMA indeed decreases the cytotoxicity but the higher the PDEAEMA block length exhibits the higher cytotoxicity. This situation limits the development of this block copolymer to be a gene carrier.

As disclosed in a master's thesis in 2005, Yang transformed the hydroxyl group (—OH) at the end of Pluronic L121 into an aldehyde group, prepared L121 as a micelle using the precipitation/solvent evaporation technique, and crosslinked the L121 micelle with the agent having an amino group (—NH₂). The L121 micelle increases the stability of a loading drug, prolongs the duration of the circulation in the body for the drug, and helps the drug entering cancer cells to inhibit their proliferation rate.

In view of the drawbacks of the prior art, the inventor has developed the present invention to overcome the drawbacks of the prior art. Introducing protonated carboxyl groups into the 2-(dimethylamino)ethyl methacrylate (DMAEMA)-based gene delivery carrier efficiently decreases the cytotoxicity and retains the high gene transfection efficiency. In addition, the tumor tissue can be targeted using a carboxyl group to react with a biomolecule. The biomolecule serves as a sensor to target a specific cancer site because of the recognition its receptors overexpressed in the tumor surface. Moreover, the biomolecule can be a therapeutic drug or a diagnostic agent to form a functionalized particle in a nanometer scale. For example, a conjugated clinical diagnosis agent is capable of functionalizing a real-time tracking agent for the molecular imaging. In another aspect, the introduction of the hydrophobic molecule increases the stability of the nanomicelle carrier in the blood circulation. The increase of the hydrophobic interaction facilitates the breakdown of the membrane of the endosome in the gene transduction and increases the transfection efficiency. The present invention broadens applications of the present carrier in nanopharmaceutics. The summary of the present invention is described below.

### SUMMARY OF THE INVENTION

The major purpose of the present invention is to provide a nanoparticle, comprising a poly(ethylene glycol)-block-poly(propylene glycol)-block-poly (ethylene glycol) (PEO-PPO-PEO) polymer compound, vinyl monomers forming a block copolymer with the PEO-PPO-PEO polymer and an active ligand conjugating the block copolymer.

According to the present invention, the nanoparticle further comprises a therapeutic drug and forms a pharmaceutical composition.

The present invention is also providing a nanoparticle, which comprises {PPEO}-{AFG}-{DV}, where {PPEO} is a poly (ethylene glycol)-block-poly (propylene glycol)-block-poly (ethylene glycol) (PEO-PPO-PEO) polymer compound; {AFG} is a vinyl monomer and {DV} is an active ligand.

A further purpose of the present invention is to provide a method for administering a pharmaceutical nanoparticle, including the steps of polymerizing an effective amount of a pharmaceutical compound with a {PPEO}-{AFG}-{DV} polymer to form the pharmaceutical nanoparticle, where {PPEO} is a poly(ethylene glycol)-block-poly(propylene glycol)-block-poly(ethylene glycol) (PEO-PPO-PEO) polymer compound; {AFG} is a vinyl monomer and {DV} is an active ligand and administering the pharmaceutical nanoparticle to a subject in need thereof.

The above poly(ethylene glycol)-block-poly(propylene glycol)-block-poly(ethylene glycol) (PEO-PPO-PEO) polymer is a tri-block copolymer formed by the polyethylene oxide and the polypropylene oxide monomer, which has a trade name of Pluronics®. Pluronics® can serve as an amphiphilic (water soluble and organic soluble) tri-block copolymer in the present invention.

Pluronics® is one selected from a group consisting of Pluronic L35, Pluronic L43, Pluronic L44, Pluronic L61, Pluronic L62, Pluronic L64, Pluronic L81, Pluronic L92, Pluronic L101, Pluronic L121, Pluronic P84, Pluronic P85, Pluronic P103, Pluronic P104, Pluronic P105, Pluronic P123, Pluronic F68, Pluronic F87, Pluronic F88, Pluronic F98, Pluronic F108, Pluronic F127 and a combination thereof.

The above vinyl monomer refers to a compound selected from a group consisting of an acrylate, an acrylamide, a methylacrylamide, a methacrylate and a combination thereof. The acrylate is one selected from a group consisting of 2-hydroxyethyl acrylate (HEA), tert-butyl acrylate (tBA), glycidyl acrylate (GA) and a combination thereof. The acrylamide is such as the dimethylacrylamide (DMAA). The methacrylate is one selected from a group consisting of 2-(diethylamino)ethyl methacrylate (DEAEMA), 2-(dimethylamino)ethyl methacrylate (DMAEMA), 2-(diisopropylamino)ethyl methacrylate (DPAEMA), (2-hydroxy-3-(2-aminoethyl)amino)propyl methacrylate (HAEAPMA), glycidyl methacrylate (GMA), poly(ethylene glycol) methacrylate (PEGMA), poly(glycidyl methacrylate) (PGMA) and a combination thereof. The methylacrylamide is one selected from a group consisting of methacryloxysuccinimide (MAS), 2-lactobionamidoethyl methacrylamide (LAEMA), N-[3-(dimethylamino)propyl]methacrylamide (DMAPMA), 2-aminoethyl methacrylate (AEMA), 3-aminopropyl methacrylamide (APMA), N-(2-hydroxyethyl)methacrylamide (HEMA), N-(2-hydroxypropyl)methacrylamide (HPMA) and a combination thereof.

According to the present invention, the active ligand itself usually has a specific bioactivity and binds to the receptor for presenting the specific bioactivity. The block copolymer designed in the present invention connects to the targeted amino acid group of the biomolecule, the antibody and the fragment thereof through its active functional group to form a detectable marker. The active functional group, for example, is the carboxyl group (—COOH), the amino group (—NH₂) or the sulfhydryl group (—SH). The active functional group (i.e. —COOH) can also reduce the cytotoxicity of the block copolymer. The present invention provides a tracking agent for the targeted tumor tissue or the molecular image, which broadens applications of the carrier in nanopharmaceutics.

According to the present invention, the active ligand is one selected from a group consisting of folic acid, arginine-glycine-aspartate (Arg-Gly-Asp, RGD) sequence, transferrin, Angiopep, chlorotoxin and a combination thereof. According to the present invention, the Angiopep is one selected from a group consisting of Angiopep-1, Angiopep-2, Angiopep-3, Angiopep-4a, Angiopep-4b, Angiopep-5, Angiopep-6, Angiopep-7 and a combination thereof.

Since the folic acid is an essential molecule in the cell growth, the surfaces of the cancer cells in mitosis have many folic acid receptors (FAR), such as the type of the glycosylphosphatidylinositol linked membrane glycoprotein α-FAR, β-FAR and γ-FAR. These FARs have highly specific expressions in various cancer cells and the dissociation constant (k_{d}) thereof is 0.1 nM. Folic acid belongs to an ideal probe in drug-targeted delivery systems. It has a comparably probing ability to the tumor tissue through the FARs-mediated endocytosis. Cholic acid is a biological detergent in the body, which undergoes the sterol nucleus modifications from the steroid and the oxidation step in its side chain, and then be released by the liver. The whole process includes complex metabolic pathways. The secreted cholic acid from the liver goes to the intestine via the bile duct and preferably helps the absorptions of the lipid and the lipid-soluble vitamin. Since cholic acid is a lipidphilic molecule, it increases the stability of the micelle carrier because of increasing hydrophobicity, and also induces endosome collapse in the gene transduction for increasing the transfection efficiency.

Arginine-glycine-aspartate (RGD) exists in various extracellular matrixes, which can specifically bind to 11 integrins (i.e. the cell adhesion receptor αᵥβ₃ integrin containing in cancer cells upon tumor angiogenesis and tumor metastasis) and facilitates the adhesion of biomaterials to the cells efficiently, and thus usually serves as an identification mediator for the cancer cells. The chemotherapeutic effect in a drug delivery system can be increased through linking transferrin on the biomaterials because the malignant tumor needs iron to produce the cytokines. Therefore, transferrin usually serves as a ligand to target tumor cells. Angiopep can be delivered to the liver, the lungs, the kidneys, the spleen and muscles. Angiopep-1, Angiopep-2, Angiopep-3, Angiopep-4a, Angiopep-4b, Angiopep-5 and Angiopep-6 can pass the blood-brain barrier (BBB) but Angiopep-7 can not. Chlorotoxin belongs to a chlorine ion channel and is non-toxic to mammals, and is able to specifically bind to a malignant sarcoma, an intestinal tumor and other tumor cells such as a prostate tumor. Thus, chlorotoxin serves as a ligand to target tumor cells.

The above polymer nanoparticle can serve as a gene carrier because of the following advantages: (1) the nanoparticle can be coated with the ribonucleotides and prevents them from degradation. (2) the nanoparticle has a high specific surface area to be linked with a specific ligand to achieve the specificity for the gene therapy, (3) the nanoparticle elongates the duration in the circulation system as compared with common particles because it can not be quickly removed by phagocytes. (4) the nanoparticle slowly releases ribonucleotides in a controlled manner to sustain an effective concentration for trasfection. (5) the nanoparticle is biocompatible and produces few metabolic products, less side effects and no immune rejection. A nano/micro biochip is manufactured on the matrix material such as glass, silicon film and plastics or a fluid system using techniques such as nano/micro particle manufacturing technology, nano/micro electronics, nano/micro machinery and nano/micro optoelectronics. The nanoparticle can be used as a product for biochemical analysis, diagnosis and treatment.

The above micellar drug carrier containing an active ligand is usually used to carry the hydrophobic drug such as nonsteroidal anti-inflammatory drugs, steroid or anti-cancer drugs. With regard to the drug-targeted delivery, there is a problem of non-selective clearance in the reticuloendothelial system (RES). The drug carrier utilizing a polymer micellar system may stably cover the drug in a hydrophobic layer of the nano micelle particle. Moreover, the nano micellar drug carrier preferably enhances permeability and retention effects in the tumor. In the embodiments, the nonsteroidal anti-inflammatory drug is preferably selected from Naproxen, Diclofenac, Indomethacin or Niflumic. The steroid is preferably selected from Fluocinolone, Betamethasone etc. The anti-cancer drug is preferably selected from Paclitaxel, Epirubicin (EPI), Doxorubicin, Camptothecin, Topotecan, Cyclosporine A, Rapamycin etc.

The above excipients or the phrases “pharmaceutically acceptable carrier or excipients” and “bio-available carriers or excipients” include any appropriate compounds known to be used for preparing the dosage form, such as the solvent, the dispersing agent, the coating, the anti-bacterial or anti-fungal agent and the preserving agent or the delayed absorbent. Usually, such a carrier or excipient does not have therapeutic activity itself. Each formulation prepared by combining the nanoparticle disclosed in the present invention and the pharmaceutically acceptable carriers or excipients will not cause an undesired effect, allergy or other inappropriate effects while being administered to an animal or human. Accordingly, the nanoparticle disclosed in the present invention in combination with the pharmaceutically acceptable carrier or excipients are adaptable in clinical uses. A therapeutic effect can be achieved using the dosage form in the present invention by local or sublingual administration via venous, oral, and inhalation routes or via nasal, rectal and vaginal routes. About 0.1 mg to 100 mg per day of the active ingredient is administered for the patients of various diseases.

The carrier is varied with each formulation, and the sterile injection composition can be dissolved or suspended in non-toxic intravenous injection diluents or solvent such as water and 1,3-butanediol. Besides, the fixing oil or the synthetic glycerol ester or di-glycerol ester is the commonly used solvent. The fatty acid such as oleic acid, olive oil or castor oil and glycerol ester derivatives thereof, especially oxy-acetylated type, preferably serve as the oil for preparing the injection and as the naturally pharmaceutical acceptable oil. Such oil solutions or suspensions preferably include long chain alcohol diluents or dispersing agents, carboxylmethyl cellulose or analogous dispersing agents. Other carriers are common surfactant such as Tween and Spans or other analogous emulsions, or pharmaceutically acceptable solid, liquid or other bio-available enhancing agents used for developing the formulation that is used in the pharmaceutical industry.

The composition for oral administration adopts any oral acceptable formulation, which includes capsule, tablet, pill, emulsion, aqueous suspension, dispersing agent and solvent. The carrier generally used in the oral formulation, taking a tablet as an example, the carrier is preferably lactose, corn starch and lubricant, and magnesium stearate is the basic additive. The excipients used in a capsule include lactose and dried corn starch. For preparing an aqueous suspension or an emulsion formulation, the active ingredient is suspended or dissolved in an oil interface in combination with the emulsion or the suspending agent, and an appropriate amount of sweetening agent, flavors or pigment is added as needed.

A nasal aerosol or inhalation composition is preferably prepared according to well-known preparation techniques. For example, bioavailability can be increased by dissolving the composition in a phosphate buffer saline and adding benzyl alcohol or other appropriate preservative, or an absorption enhancing agent. The nanoparticle of the present invention is preferably formulated as suppositories for rectal or virginal administration.

The nanoparticle of the present invention also can be administered intravenously, as well as subcutaneously, parentally, muscular, or by the intra-articular, intracranial, intra-articular fluid and intraspinal injections, aortic injection, sterna injection, intra-lesion injection or other appropriate administrations.

Other objects, advantages and efficacies of the present invention will be described in detail below taken from the preferred embodiments with reference to the accompanying drawings, in which:

### BRIEF DESCRIPTION OF THE DRAWINGS

FIGS. 1(A)-(C) show the Fourier transform-infrared spectrum of (A) PF127-p (DMAEMA), (B) PP123-p (DMAEMA) and (C) PL121-p (DMAEMA).

FIG. 2 shows the critical micelle concentrations of PF127-p (DMAEMA), PP123-p (DMAEMA) and PL121-p (DMAEMA).

FIG. 3 shows the acid/base titration profiles of the block copolymers of the present invention.

FIG. 4 shows the acid/base titration profiles of the modified block copolymers of the present invention.

FIG. 5 shows the particle sizes of the polyplexes formed of low, medium and high molecular weight DMAEMA block copolymers complexed with DNA.

FIG. 6 shows the zeta potentials of the modified block copolymer/DNA polyplexes.

FIG. 7 shows the cytotoxicities of the polyplexes formed of low, medium and high molecular weight block copolymers complexed with DNA.

FIG. 8 shows the cytotoxicities of the modified block copolymers.

FIG. 9 shows the transfection efficiencies of the modified block copolymer/DNA polyplexes in the absence of the serum.

FIG. 10 shows the transfection efficiencies of the modified block copolymer/DNA polyplexes in the serum.

FIG. 11 shows the cytotoxicities of the block copolymers.

FIG. 12 shows the IC₅₀ values of the EPI encapsulated by the nanoparticle at 24 hrs.

FIG. 13 shows the IC₅₀ values of the EPI encapsulated by the nanoparticle at 48 hrs.

### DETAILED DESCRIPTION OF THE PREFERRED EMBODIMENTS

Further embodiments herein may be formed by supplementing an embodiment with one or more element from any one or more other embodiment herein, and/or substituting one or more element from one embodiment with one or more element from one or more other embodiment herein.

### Examples

The following non-limiting examples are provided to illustrate particular embodiments. The embodiments throughout may be supplemented with one or more detail from one or more example below, and/or one or more element from an embodiment may be substituted with one or more detail from one or more example below.

Pluronic® PL121, PP123 and PF127 serve as the main skeleton of the block copolymer of the present invention and are used to polymerize with a cationic monomer. Among three Pluronic® derivatives, PF127 has the highest molecular weight, and the lengths of the PPO hydrophobic blocks of three Pluronic® derivatives are close while those of the PEO hydrophilic blocks vary. As shown in Table 1, the hydrophile-lipophile balance number (HLB) demonstrates various hydrophilic/lipophilic ratios.

TABLE 1

| | | | | Molecular |
|---|---|---|---|---|
| | | | | weight |
| Pluronic | PPO | PEO | HLB | (M_{w}) |
| PL121 | 68 | 10 | 1 | 4400 |
| PP123 | 70 | 40 | 8 | 5800 |
| PF127 | 65 | 200 | 22 | 12600 |

Pluronic® PL121, PP123 and PF127 are modified with a bromo group at their respective hydroxyl group at the end to serve as polymerization reactive sites. The structures of the modified Pluronic® PL121, PP123 and PF127 are determined with ¹H-NMR, where 1.90 ppm shows the characteristic peak of 2-bromoisobutyryl-CH₃. Grafting ratios of Pluronic® PL121, PP123 and PF127 are 98%, 95% and 94%, calculated from the integral values of 1.15 ppm propylene oxide-CH₃ of Pluronic® and 1.90 ppm 2-bromoisobutyryl-CH₃. The result proves that Pluronic® has been successfully modified with the bromo group.

The present invention applies the atom transfer radicals polymerization (ATRP) developed by Matyjaszewski et al., J. Am Chem. Soc. 117:5614-15, 1995. The Pluronic polymers are copolymerized with a fixed amount of 2-(dimethylamino)ethyl methacrylate (DMAEMA) monomer, and the copper ion (1+) activates the end of Pluronic®-Br and reacts with the double bonds of DMAEMA via an addition polymerization in order to prepare the {PPEO}-pDMAEMA block copolymers. PPEO represents Pluronic® PL121, PP123 and PF127.

The Pluronic polymer derivative may also copolymerized with various amounts of 2-(dimethylamino)ethyl methacrylate (DMAEMA) monomer, and the CuBr/2,2′-bipyridine activate the end of Pluronic®-Br and reacts with the double bonds of the DMAEMA for the free-radical polymerization in order to prepare the {PPEO}-pDMAEMA block copolymers with different DMAEMA block lengths of the above Pluronic® PL121, PP123 and PF127.

The NMR spectra of the block copolymers exhibit peaks at 2.26 ppm (N—CH₃), 2.70 ppm (N—CH₂) and 4.40 ppm (O—CH₂), which are attributed to the pDMAEMA. The length of the polymerized block of the DMAEMA repeating unit, being as the degree of polymerization (DP) thereof, is calculated from the integral value of 1.15 ppm attributed to the propylene oxide-CH₃ of Pluronic® and 2.26 ppm attributed to N—CH₃ of pDMAEMA. The DPs of pDMAEMA in the PL121-b-pDMAEMA, PP123-b-pDMAEMA and PF127-b-pDMAEMA are 33, 34 and 38, respectively.

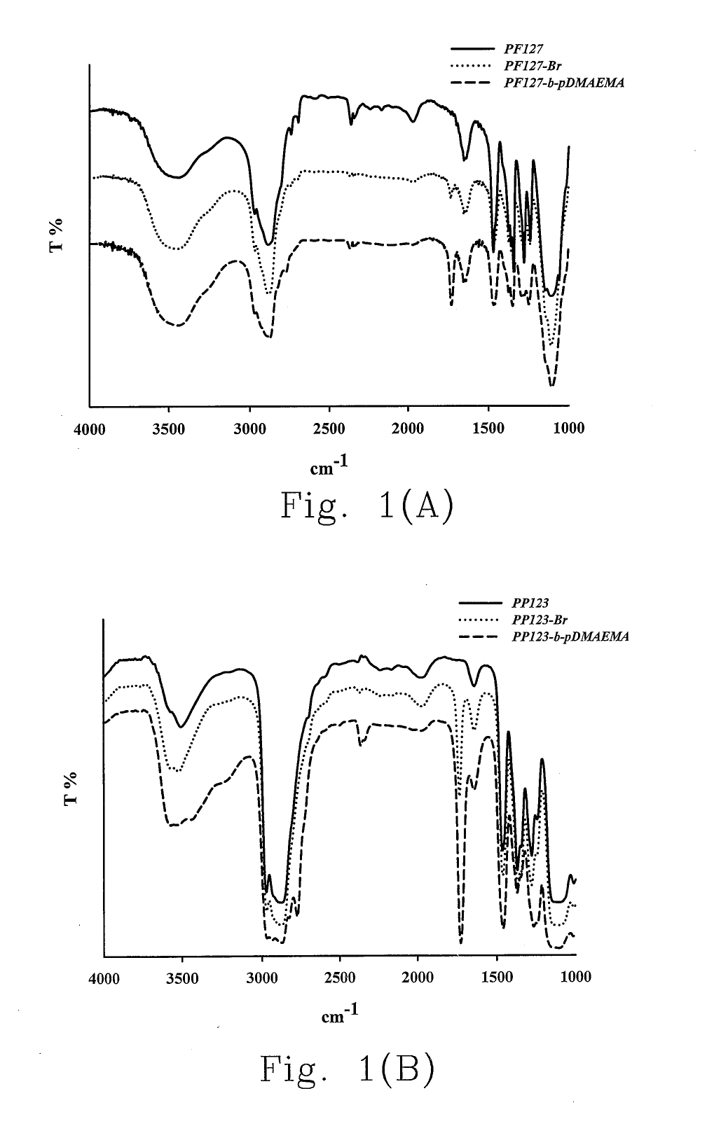

As shown in the Fourier transform infrared spectroscopy (FT-IR) spectrum of FIG. 1, the nascent Pluronic® does not display C═stretching, while the absorption band due to C═O stretching appears at 1726 cm⁻¹ after the Pluronic® are modified with 2-bromoisobutyryl as a reactive agent (Pluronic®-Br). The spectrum of the block copolymer shows characteristic peaks of the DMAEMA at 1726 cm⁻¹ (C═O), 2767 cm⁻¹ and 2819 cm⁻¹ (N—CH₃). The absorption bands of the pDMAEMA at 1726 cm⁻¹ and 2941 cm⁻¹ apparently increase with increasing the degree of polymerization. The above results prove the structures of Pluronic®-Br and their block copolymers.

The molecular weight of the block copolymers is measured using the gel permeation chromatography (GPC). The polydispersity index (PDI) of the PF127-pDMAEMA block copolymers ranges between 1.3 and 1.5, and the molecular weights are about 14000˜28000 g/mol. Among three block copolymers, the molecular weights are 13847 g/mol, 21074 g/mol, and 28632 g/mol, respectively, to the low block copolymer (PF127-pDMAEMA-L), the medium block copolymer (PF127-pDMAEMA-M) and the high molecular weight block copolymer (PF127-pDMAEMA-H).

TABLE 2
DP of the PF127-pDMAEMA and PD127-p(DMAEMA-tBA)

| | | | (DMAEMA) | | DP^{NMR} |
|---|---|---|---|---|---|
| Sample | Mₙ^{GPC} | PDI^{GPC} | DP^{GPC} | DP^{NMR} | (tBA) |
| PF127 | 10220 | 1.45 | | | |
| PF127-[A] | 28632 | 1.34 | 118 | 135 | |
| PF127-[B] | 27123 | 1.40 | | 140 | 15 |

Note:
average molecular weight (Mₙ^{GPC}) of the gel filtration chromatography (GPC)
Polydispersity index of the GPC (PDI^{GPC})
DP of the DMAEMA shown by the GPC (DP^{GPC} (DMAEMA))
DP of the DMAEMA displayed by NMR (DP^{NMR} (DMAEMA))
DP of the tBA displayed by NMR (DP^{NMR} (tBA))
PF127-[A] is PF127-pDMAEMA
PF127-[B] is PF127-p(DMAEMA-tBA)

The tBA monomer is used to participate the polymerization of the PF127-pDMAEMA block polymer to form the PF127-p(DMAEMA-tBA). From the ¹H-NMR spectrum, the characteristic peak of tert-butyl C—(CH₃)₃ group appears at 1.4 ppm. The DPs of the pDMAEMA and PtBA as shown in Table 2 are calculated and compared with the value measured by GPC. The PF127-p(DMAEMA-tBA) has the molecular weight of 27123 g/mol with a narrow molecular weight distribution (PDI=1.40). Additionally, the ¹H-NMR spectrum shows that the tert-butyl C—(CH₃)₃ characteristic peak of the PF127-p(DMAEMA-tBA) originally at 1.4 ppm disappears after the hydrolysis. The characteristic peaks of the DMAEMA at 2.3 ppm (N—CH₃) and 2.6 ppm (N—CH₂) shift to 2.9 ppm (N—CH₃) and 3.2 ppm (N—CH₂) because of the protonation. From the FT-IR observation, the hydroxyl group (—OH) characteristic peak appears at 3200˜3600 cm⁻¹ after hydrolysis. Taken together, it is proved that the PF127-p(DMAEMA-AA) has been synthesized.

Since human blood contains 70% water, the concentration of the carrier may be greatly diluted after the carrier enters the circulation. When the concentration of the carrier is lower than the critical micelle value thereof, the carrier will disassemble into a unimer and thus is impossible to protect the carried drug in the circulation system.

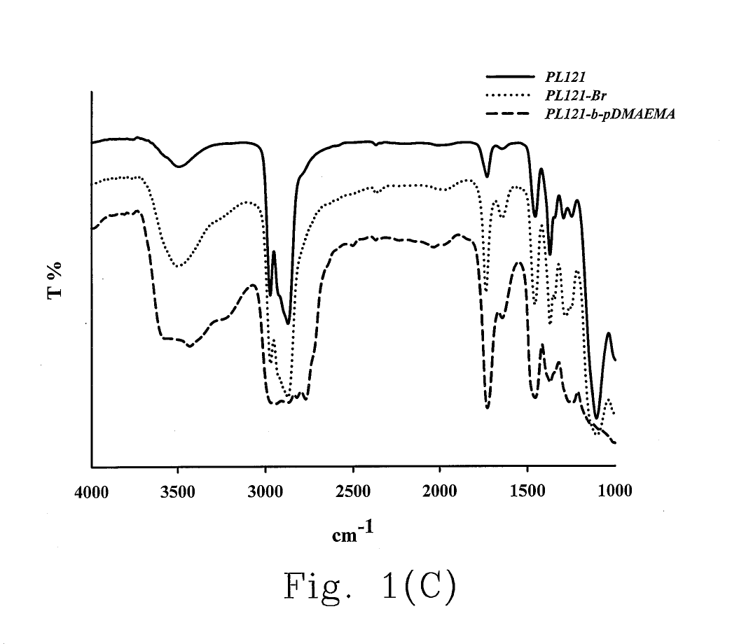

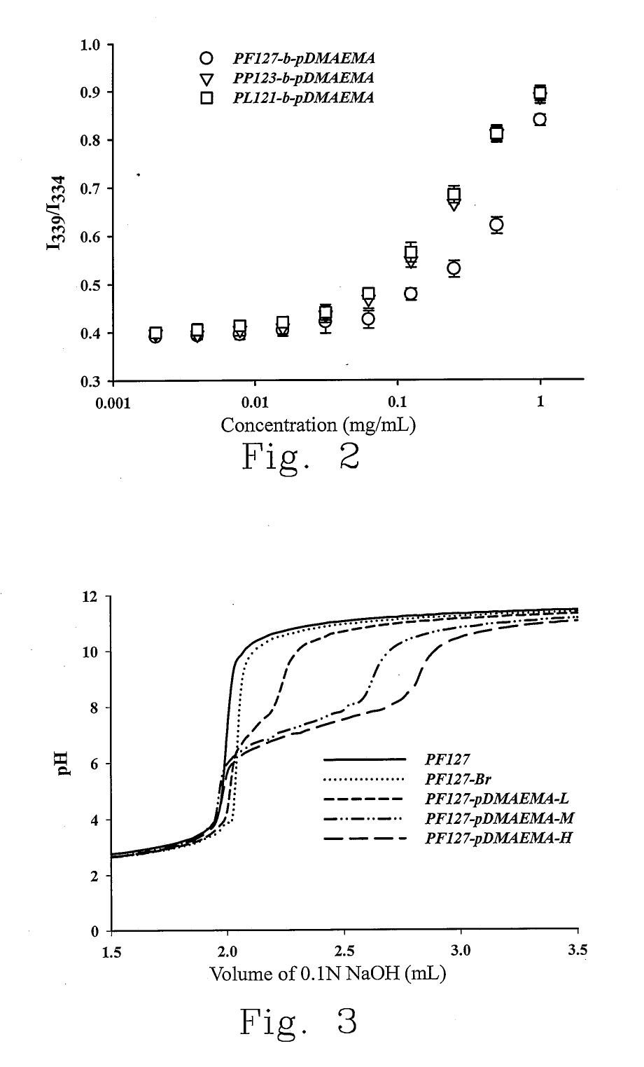

Pluronic® polymer has the ability to self-assemble into a micelle when the polymer concentration is controlled at a concentration of higher than the critical micelle concentration (CMC). The Pluronic® polymer materials with three different hydrophilic/lipophilic ratios are regulated using the copolymer concentrations to encapsulate pyrene. The vibronic band intensity of pyrene is sensitive to the solution polarity. The CMC of three block copolymers are studied by measuring the ratio of the first (I₁) and the third (I₃) vibronic bands (I₁/I₃) of pyrene. As shown in Table 1, the PPO hydrophobic blocks of PL121, PP123 and PF127 have similar length of 68, 70 and 65 respectively, so that the difference of the hydrophilic-lipophilic character is based on the lengths of the PEO blocks being as 10, 40 and 200, respectively. As shown in FIG. 2 and Table 3, the CMC values of PL121, PP123 and PF127 are 4.40×10⁻³ mg/mL, 2.53×10⁻² mg/mL and 3.53×10⁻² mg/mL, and the CMC values increase after being polymerized with the hydrophilic pDMAEMA.

TABLE 3

| | critical micelle concentration |
|---|---|
| Sample | (CMC) |
| PL121 | 4.40 × 10⁻³ mg/mL |
| PP123 | 2.53 × 10⁻² mg/mL |
| PF127 | 3.53 × 10⁻² mg/mL |
| PL121-b-pDMAEMA | 3.54 × 10⁻² mg/mL |
| PP123-b-pDMAEMA | 3.64 × 10⁻² mg/mL |
| PF127-b-pDMAEMA | 4.99 × 10⁻² mg/mL |

Another purpose of the present invention is to increase the hydrophobic character of the block copolymer for increasing stability of the drug carrier through introducing an active functional group which binds to the hydrophobic molecule. From the above data, it is known that the smaller the CMC value, the higher the hydrophobic property, and thus the higher efficiency of encapsulating a hydrophobic drug. Cholic acid is introduced to increase the hydrophobicity as shown in Table 4.

TABLE 4
Critical micelle concentration

| Sample | CMC |
|---|---|
| PF127 | 3.53 × 10⁻² mg/mL |
| PF127-pDMAEMA | 7.7 × 10⁻¹ mg/mL |
| (high molecular weight) | |
| PF127-b-pDMAEMA | 4.99 × 10⁻² mg/mL |
| (low molecular weight) | |
| PF127-p(DMAEMA-co-AMA-CA) | 1.8 × 10⁻² mg/mL |
| (high molecular weight) | |

The camptothecin-encapsulated micelle using the high molecular weight block copolymer PF127-pDMAEMA-H shows 15.45% encapsulation efficiency (EE %) and 1.7% loading efficiency (LE %), and that using the cholic acid-introduced PF127-p(DMAEMA-co-AMA-CA) shows 34.7% EE % and 3.817% LE %, respectively. These data prove that the encapsulation efficiency of the block copolymer can be increased by introducing hydrophobic cholic acid.

The micelle is prepared by emulsification and the hydrophobic anti-cancer drug is encapsulated at the inner core of the Pluronic® hydrophobic block. Table 5 shows the EE % and LE % of Epirubicin (EPI) encapsulated with the {PPEO}-pDMAEMA block copolymer of Pluronic® PL121, PP123 and PF127.

TABLE 5

| drug/ | | Encapsulation | Loading |
|---|---|---|---|
| micelle | | efficiency | efficiency |
| (wt %) | Micelle | (EE %) | (LE %) |
| 10 wt % | PL121-b-pDMAEMA | 71.7940 ± 5.6520 | 6.5267 ± 0.5138 |
| | PP123-b-pDMAEMA | 78.6627 ± 10.627 | 7.1512 ± 0.9662 |
| | PF127-b-pDMAEMA | 68.1339 ± 11.285 | 6.1940 ± 1.0260 |

Tetrahydrofuran (THF) is used as dispersing agent to prepare the micelle, and the volume ratio of the THF and water is 1:10. The micelle displays clear and no precipitation or suspension when it disperses in water at a concentration of 1 mg/mL. In Table 6, the particle distribution and zeta potential of the micelle formed by the block copolymer are analyzed using the dynamic light scattering (DLS). The average particle sizes of the Pluronic-pDMAEMA (PF127-b-pDMAEMA, PP123-b-pDMAEMA and PL121-b-pDMAEMA) are 206.9±9.522 nm, 271.0±30.01 nm and 350.9±24.48 nm, respectively, and the particle surfaces of three different Pluronic-pDMAEMA copolymers are positive-charged. The particle size exhibits an increased trend if the hydrophobic characteristic of the block copolymer micelle increases. This is due to the ratio of the PEO block in the micelle decreasing and the PPO becoming the main structure of the micelle, and thus leading to a larger particle size. This result is in agreement with the finding by Ge, H. et al. (J. Pharm. Sci. 910(6):1463-73, 2002). They reported that the micelle particle size exhibits a decreased trend along with the increased ratio of the PEG when the end of PCL-PEO-PCL is modified with various ratios of PEGs.

Due to the presence of hydrophobic force between the hydrophobic drug and the hydrophobic PPO block of Pluronic®-pDMAEMA, the average micelle particle sizes of three different kinds of Pluronic®-pDMAEMA decrease after carrying the EPI. Among the three different kinds of Pluronic®-pDMAEMA, PL121-b-pDMAEMA having the highest hydrophobic property has a significant change in particle size, the average particle size decreases from 351 nm (unloaded) to 157 nm. The zeta potentials of the three different kinds of Pluronic®-pDMAEMA decrease significantly after carrying the drug.

TABLE 6
Analyses of the particle size distribution and the
zeta potential of the carrier micelles (n = 3)

| | | | Zeta |
|---|---|---|---|
| | Particle size | | potential |
| Micelle | (nm) | PDI | (mV) |
| (A) PL121-b- | 350.9 ± 24.48 | 0.317 ± 0.0323 | 18.7 ± 0.65 |
| pDMAEMA | | | |
| (A) PP123-b- | 271.0 ± 30.01 | 0.333 ± 0.0282 | 21.5 ± 0.62 |
| pDMAEMA | | | |
| (A) PF127-b- | 206.9 ± 9.522 | 0.287 ± 0.0253 | 19.1 ± 0.51 |
| pDMAEMA | | | |
| (B) PL121-b- | 157.5 ± 8.273 | 0.303 ± 0.0095 | 7.07 ± 0.65 |
| pDMAEMA | | | |
| (B) PP123-b- | 140.0 ± 3.109 | 0.240 ± 0.0025 | 9.97 ± 0.62 |
| pDMAEMA | | | |
| (B) PF127-b- | 172.2 ± 4.900 | 0.252 ± 0.0055 | 13.0 ± 0.51 |
| pDMAEMA | | | |

Note:
(A) unloaded
(B) Epirubicin loaded

The micelle is prepared at 1 mg/mL and dispersed in water, and the micelle morphology is observed using a transmission electron microscope (TEM). The micelle is positive-charged because of the pDMAEMA. This prevents the micelle aggregation. Moreover, Pluronics® is an amphiphilic polymer that can self-assemble into a micelle structure; therefore these three block copolymers display core-shell morphology. The hydrophilic/lipophilic balance causes the different core sizes. The highest lipophilic PL121-b-pDMAEMA has the largest core while the highest hydrophilic PF127-b-pDMAEMA has the most apparent shell. The micelle particle sizes of PF127-b-pDMAEMA, PP123-b-pDMAEMA and PL121-b-pDMAEMA are 200 nm, 210 nm and 240 nm. The observed particle size is less than the value measured by DLS due to the determination of the DLS in water accounting for the swelling diameter of the micelle.

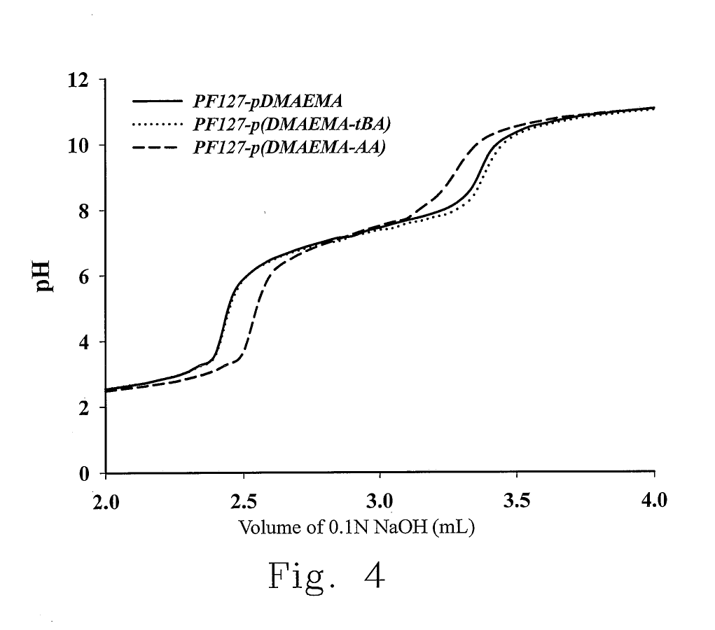

Since the cationic polymers preferably induce the breakdown of the endosome using proton sponge effect and achieve gene transfer efficiency, the buffering capacity of a gene carrier also plays a role in gene transfection efficiency. The buffering capacity of the carrier is determined using the acid-base titration. After adding equal volume of the acid or base, the smaller pH change of the different carriers shows the better buffering effect. As shown in FIG. 3, the PF127 and PF127-Br have no buffering capacity, and the buffering capacity of the PF127-pDMAEMA block copolymer increases with the block length of pDMAEMA because more NaOH is needed to change a certain pH range. Therefore, the PF127-pDMAEMA-H has the best buffering capacity and a smooth titration curve. Compared with the titration curve of the PF127-pDMAEMA, the PF127-p(DMAEMA-tBA) shows no significant change. The PF127-p(DMAEMA-AA) formed after tBA hydrolyzed induces a more deprotonation and thereby decreases the buffering effect of the pDMAEMA. Since the positive charges of the pDMAEMA also result in the cytotoxicity, an appropriate amount of introduced acrylic acid (AA) can decrease the cytotoxicity caused by the positive charges. This situation, at the same time, increases the biocompatibility of PF127-p(DMAEMA-AA) as a gene carrier, although the buffering capacity of the pDMAEMA is slightly sacrificed. As shown in FIG. 4, PF127-p(DMAEMA-AA) still retains a good buffering capacity and can be used as a gene delivery carrier.

To investigate the influence of the DMAEMA block length on the ability to protect DNA, PF127-pDMAEMA with three different DMAEMA block lengths (as shown in Table 7, i.e. the low molecular weight PF127-pDMAEMA-L, the medium molecular weight PF127-pDMAEMA-M and the high molecular weight PF127-pDMAEMA-H) are prepared for testing the-DNA protecting ability.

The DNA passes through the gel and move toward the positive electrode using its negative charged property. The DNA-biding ability of the different cationic polymers under various Nitrogen/Phosphate (N/P) ratios can be observed by the gel electrophoresis/retardation assay after the ethidium bromide staining. Since the polyplex is prepared with the electrostatic interactions between the cationic polymers and negative-charged DNA, it will be interfered by the charged protein, which may cause the DNA dissociation from the polyplex when delivered into the body. In order to observe whether the PF127-pDMAEMA/DNA polyplex can resist the effect of the serum, a total concentration of 10% fetal bovine serum (FBS) is supplemented into the formulation used to prepare the polyplex for testing the DNA-protecting ability of the carrier.

TABLE 7

| Sample | Mₙ^{GPC} | PDI | DP^{GPC} | DP^{NMR} | Yield (%) |
|---|---|---|---|---|---|
| PF127 | 10220 | 1.45 | | | |
| PF127-[L] | 13847 | 1.40 | 24 | 34 | 65 ± 5 |
| PF127-[M] | 21074 | 1.33 | 70 | 72 | 63 ± 7 |
| PF127-[H] | 28632 | 1.34 | 118 | 135 | 57 ± 8 |

Note:
the average molecular weight of the GPC (Mₙ^{GPC})
the polydispersity index (PDI)
DP shown by the GPC (DP^{GPC})
DP shown by the NMR (DP^{NMR})
PF127-[L] is a low molecular weight PF127-pDMAEMA-L
PF127-[M] is a medium molecular weight PF127-pDMAEMA-M
PF127-[H] is a high molecular weight PF127-pDMAEMA-H

The polyplexes formed of the PF127-pDMAEMA-L, PF127-pDMAEMA-M and PF127-pDMAEMA-H block copolymers complexed with DNA under serum-free condition are analyzed by gel electrophoresis, wherein the N/P ratios of the gel electrophoresis and DNA are 1, 3, 6, 9 and 12. When the N/P ratio is 1, all three pDMAEMAs can efficiently encapsulate the DNA. Three pDMAEMAs do not show the naked DNA in serum. The gel electrophoresis images of the pDMAEMAs/DNA in serum are similar to those in serum-free condition. Accordingly, PF127-p(DMAEMA-tBA) also shows the result similar to PF127-pDMAEMAs when complexed with DNA. Although PF127-p(DMAEMA-AA) has a carboxyl group, the carboxyl group does not affect the encapsulating ability and stability of the DNA.

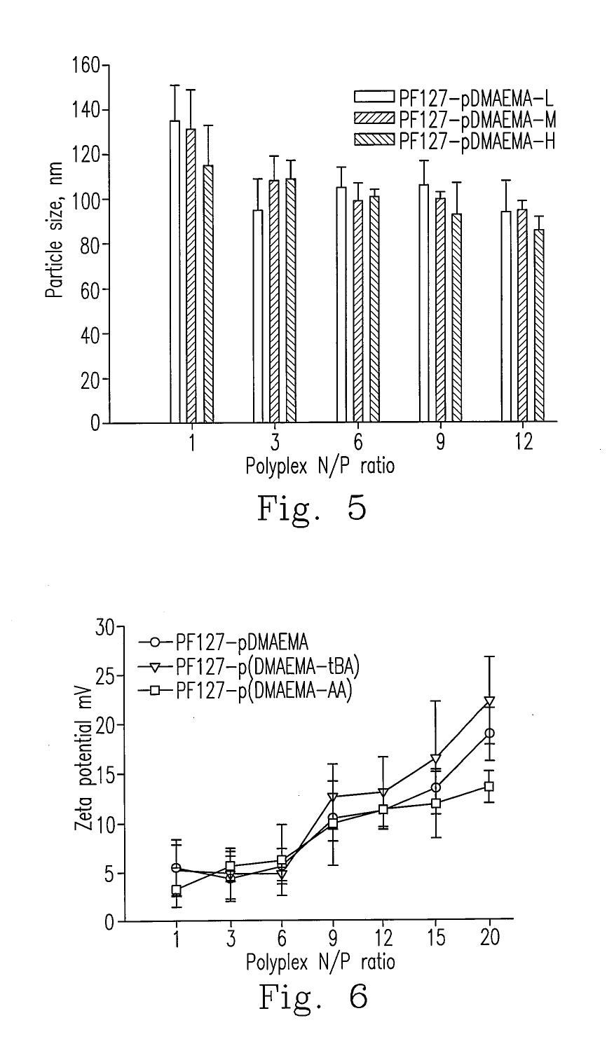

The cationic polymer and the plasmid DNA are mixed in the solution and form a polyplex through the electrostatic interactions between the positive and negative charges. Theoretically, the polyplex enters the cells through the endocytosis and forms the endosome. The endosome releases the plasmid, and the plasmid passes through the cytoplasm to the nuclei and performs the gene transfection. In FIG. 5, the particle sizes of the polyplexes with three different pDMAEMA block lengths are within 110˜140 nm. When the N/P ratio increases, the structure of the polyplex is more compact and the particle size decreases to 80˜100 nm.

A cationic polymer bears positive charges in the mild acidic environment due to the protonation, which enables it to interact with the negative-charged DNA to form a polyplex. From the zeta potential analysis, the potential of the polyplex is tend to be a positive value (about 10˜15 mV), and the electrophoresis test of the PF127-pDMAEMA/DNA polyplexes also indirectly proves that the DNA is well encapsulated. All of the modified block copolymers do not affect the encapsulation efficiency of the DNA. The particle sizes of the PF127-pDMAEMA/DNA and PF127-p(DMAEMA-tBA)/DNA polyplexes are within 120˜170 nm and the particle size of the PF127-p(DMAEMA-AA)/DNA polyplex is 90˜150 nm. It is presumed that the PF127-p(DMAEMA-AA)/DNA polyplex containing a carboxyl group can interact with the pDMAEMA block and causes a more compact polyplex structure. In FIG. 6, the zeta potentials of the PF127-p(DMAEMA-tBA)/DNA and PF127-p(DMAEMA-AA)/DNA polyplexes are on the low side, it is believed that the PEO hydrophilic block in the Pluronic® causes the shielding effect and lets the zeta potential decrease. When the PtBA and polyacrylic acid (PAA) blocks are introduced, they also affect the zeta potential thereof, and the carboxyl groups of the PAA block preferably reduce the positive charge of the pDMAEMA block and decrease the zeta potential significantly.

Gel electrophoresis shows that the encapsulation efficiency and the particle size distribution of three carrier materials display much significant difference at N/P=12, so the polyplex of N/P=12 is chosen as the main object to be observed by the TEM. By TEM observation, all polyplexes with different pDMAEMA block lengths have an approximate spherical shape. It is assumed that the Pluronic® containing PPO block has a hydrophobic effect in addition to the electrostatic force of the polyplex itself, which benefits core-shell morphology. The enhanced hydrophilicity after introducing the PAA block results in a clear observation of the shell layer in the PF127-p(DMAEMA-AA)/DNA polyplex by TEM.

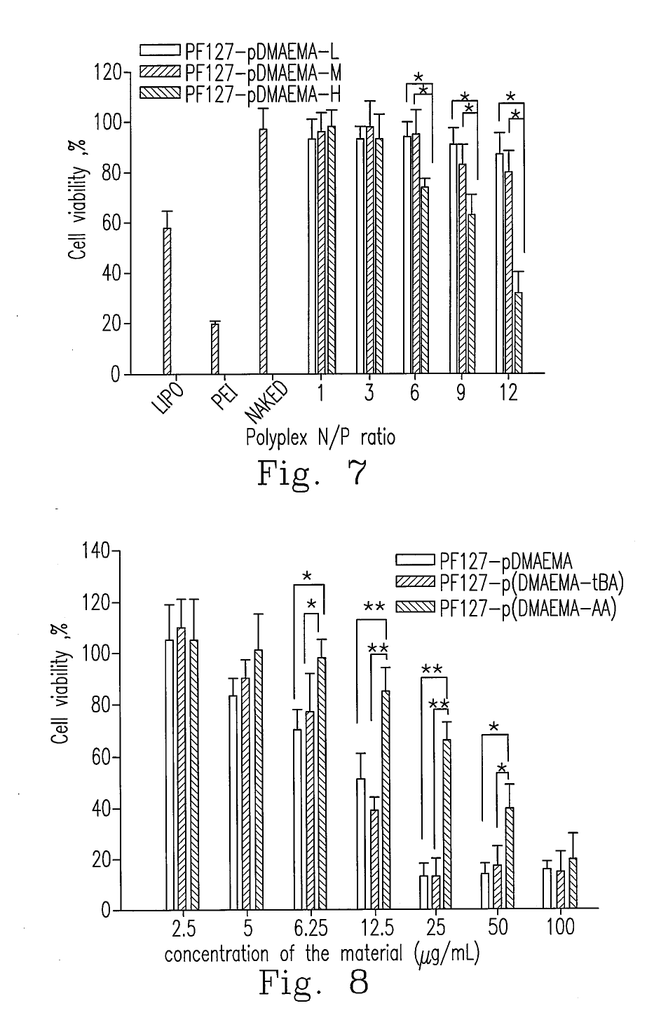

The cytotoxicity of cationic polymers causes an application limitation although they have a good gene transfection efficiency. An MTT assay is used to analyze whether the modification of the Pluronic® lowers cytotoxicity. In FIG. 7, a commercial available liposome transfection agent (Lipofectamine 2000, LIPO), polyethylenimine (PEI 25K, PEI) and the simple DNA without being protected by any carrier (naked DNA) are used as positive and negative control groups of experiments, wherein PEI 25K is a PEI having 25000 average molecular weight and N/P ratio=10.

As shown in FIG. 7, the pDMAEMA block with a high molecular weight still has the cytotoxicity. After being formed the polyplex with the DNA, the cytotoxicity of the polyplex induced by the positive charge can be reduced under the influence of the negative charge of the DNA. When the N/P ratio is over 6, the pDMAEMA block including the low and medium molecular weights show cell viability to be above 80%, but the high molecular weight pDMAEMA block still shows high cytotoxicity.

Conversely, the high molecular weight pDMAEMA block has high gene expression but causes cytotoxicity as well. In the cell transfection experiment, it shows that the naked DNA as the negative control has a limited transfection efficiency. Both of the low and medium molecular weight PF127-pDMAEMA blocks increase fluorescence expressions with the increasing N/P ratio, and the high molecular weight pDMAEMA block exhibits an opposite trend. The decreased fluorescence expression is due to that the high molecular weight pDMAEMA block causes cytotoxicity.

The effect of the modified block copolymers, PF127-p(DMAEMA-tBA) and PF127-p(DMAEMA-AA), on cytotoxicity is studied using the MTT assay (FIG. 8). Comparing with PF127-pDMAEMA, PF127-p(DMAEMA-tBA) has a similar cytotoxicity but PF127-p(DMAEMA-AA) is significantly lower. The cells remain at 100% viability when PF127-p(DMAEMA-AA) is used at a concentration of 6.25 μg/ml, but the cell viabilities of PF127-p(DMAEMA-tBA) and PF127-pDMAEMA are lower than 80%. The decrease in cytotoxicity of PF127-p(DMAEMA-AA) has a statistical significance (p<0.05) as compared with PF127-p(DMAEMA-tBA) and PF127-pDMAEMA. When the concentration is raised up to 12.5 μg/ml, the cell viability of PF127-p(DMAEMA-AA) is still greater than 80% while those of PF127-p(DMAEMA-tBA) and PF127-pDMAEMA are less than 50%. These data also show a statistically significant difference (p<0.01). When the concentration is raised up to 50 μg/ml, the cell viability of PF127-p(DMAEMA-AA) is less than 50%.

The N/P ratio is fixed at 9 and various DNA dosages are added into the polyplex to investigate the effect of the DNA concentration on cytotoxicity and gene transfection. Although the high does DNA shows the high expression of transfected genes, the cytotoxicity thereof also significantly increases. As shown in Table 8, the transfected gene expression of the PF127-p(DMAEMA-AA)/DNA polyplex is not as good as those of the PF127-pDMAEMA/DNA and PF127-p(DMAEMA-tBA)/DNA, but PF127-p(DMAEMA-AA)/DNA has lower cytotoxicity. The higher the N/P ratio, the higher the cytotoxicity as shown in Table 9. When the N/P ratio is higher than 12, the cell viabilities of the PF127-pDMAEMA/DNA and PF127-p(DMAEMA-tBA)/DNA polyplexes decrease below 50% while that of the PF127-p(DMAEMA-AA)/DNA polyplex remains at approximately 60%. It can be concluded that the modification of the PF127-p(DMAEMA) with PAA can efficiently raise the biocompatibility.

TABLE 8
The cytotoxicities of the materials

| Dose (μg/mL) | PF127-[A] | PF127-[B] | PF127-[C] |
|---|---|---|---|
| 2.5 | 105 ± 14(%) | 110 ± 11(%) | 104 ± 16(%) |
| 5 | 83 ± 7(%) | 90 ± 8(%) | 101 ± 14(%) |
| 6.5 | 70 ± 8(%) | 77 ± 15(%) | 98 ± 7(%) |
| 12.5 | 51 ± 10(%) | 39 ± 5(%) | 85 ± 9(%) |
| 25 | 13 ± 5(%) | 13 ± 7(%) | 66 ± 8(%) |
| 50 | 14 ± 4(%) | 17 ± 8(%) | 40 ± 9(%) |
| 100 | 16 ± 3(%) | 15 ± 8(%) | 20 ± 10(%) |

Note:
PF127-[A] PF127-pDMAEMA
PF127-[B] PF127-p(DMAEMA-tBA)
PF127-[C] PF127-p(DMAEMA-AA)

TABLE 9
Cytotoxicities of the polyplexes

| N/P ratio | PF127-[A]/DNA | PF127-[B]/DNA | PF127-[C]/DNA |
|---|---|---|---|
| 1 | 100 ± 15(%) | 98 ± 14(%) | 99 ± 13(%) |
| 3 | 91 ± 9(%) | 92 ± 16(%) | 96 ± 8(%) |
| 6 | 87 ± 8(%) | 81 ± 7(%) | 95 ± 9(%) |
| 9 | 72 ± 7(%) | 65 ± 9(%) | 85 ± 7(%) |
| 12 | 56 ± 6(%) | 46 ± 6(%) | 70 ± 5(%) |
| 15 | 30 ± 8(%) | 32 ± 5(%) | 54 ± 7(%) |
| 20 | 22 ± 9(%) | 20 ± 6(%) | 45 ± 5(%) |

Note:
Lipofectamine (LIPO): 58 ± 7(%)
Polyethylenimine (PEI): 19 ± 2(%)
Simple DNA: 97 ± 8(%)

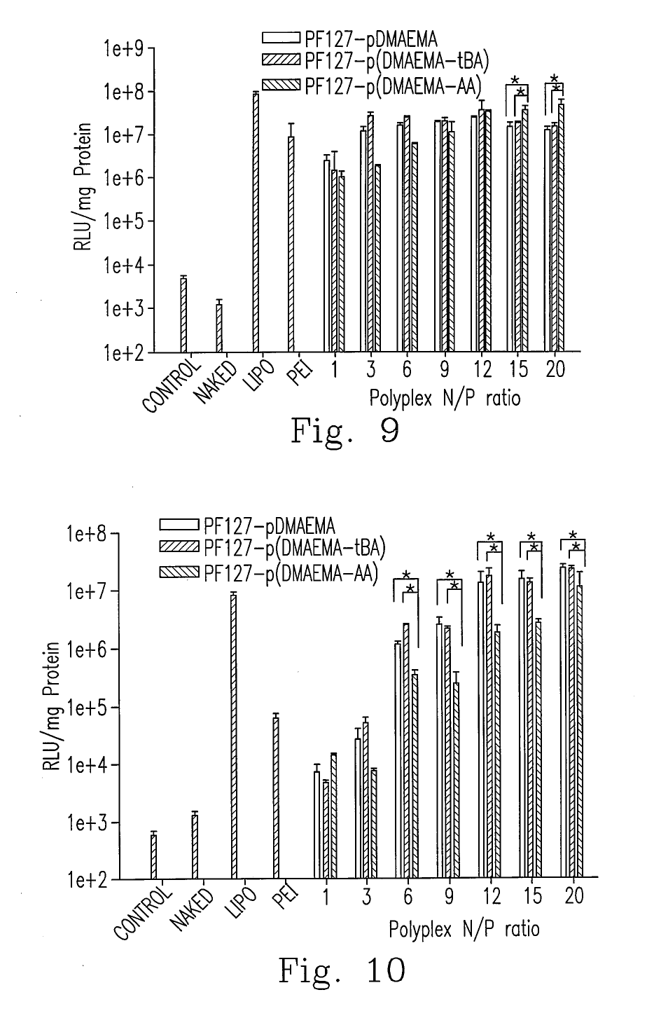

At a low N/P ratio (1-9), the gene transfection efficiency of the PF127-p(DMAEMA-AA)/DNA polyplex is less than that of the PF127-pDMAEMA/DNA and PF127-p(DMAEMA-tBA)/DNA polyplexes (FIG. 9). When the N/P ratio is increased to 12˜20, the PF127-pDMAEMA/DNA and PF127-p(DMAEMA-tBA)/DNA polyplexes induce cytotoxicity thereby decrease the gene expression. The PF127-p(DMAEMA-AA)/DNA polyplex has the enhanced gene transfection efficiency because of its better biocompatibility than PF127-pDMAEMA/DNA and PF127-p(DMAEMA-tBA)/DNA. Nevertheless, in the presence of serum as shown in FIG. 10, the transfection efficiency of the PF127-p(DMAEMA-AA)/DNA polyplex is not as good as those of the PF127-pDMAEMA and PF127-p(DMAEMA-tBA)/DNA polyplexes.

Under mild conditions, the polyplex has an excellent stability while it is interfered with the charged protein when it is applied in a human body. Thus, the 10% fetal bovine serum is used to simulate the protein competition with DNA. Table 10 shows the impact of duration on the particle sizes of the polyplexes. The PF127-pDMAEMA/DNA, PF127-p(DMAEMA-tBA)/DNA and PF127-p(DMAEMA-AA)/DNA polyplexes have no significant change in particle size distribution before adding the serum, but the particle sizes of the polyplexes change under the influence of the serum protein, wherein the PF127-pDMAEMA/DNA and PF127-p(DMAEMA-AA)/DNA polyplexes have more significant changes. It is believed that the protein can bind to the cationic pDMAEMA block as well as the anionic PAA block, leading to form the aggregation. The PF127-p(DMAEMA-tBA)/DNA polylpex has no significant difference in particle size distribution in the presence and absence of the serum protein because the hydrophobic block PtBA is contained therein and the stability is raised by the hydrophobic force.

TABLE 10
Analysis of the polyplex stability

| (N/P = 12) | PF127-[A] | PF127-[B] | PF127-[C] |
|---|---|---|---|
| Initial time (0 h) | 102 ± 11 nm | 97 ± 8 nm | 82 ± 7 nm |
| PDI | 0.289 ± 0.025 | 0.302 ± 0.038 | 0.268 ± 0.015 |
| The 4^{th} hour (4 h) | 103 ± 10 nm | 108 ± 9 nm | 92 ± 5 nm |
| PDI | 0.273 ± 0.019 | 0.264 ± 0.027 | 0.248 ± 0.021 |
| The 4^{th} hour in | 225 ± 30 nm | 142 ± 15 nm | 284 ± 47 nm |
| serum | | | |
| PDI | 0.354 ± 0.107 | 0.331 ± 0.085 | 0.407 ± 0.121 |

Note:
polydispersity index: PDI
Initial time is Time 0 hr, which simply referred to as 0 h
The 4^{th} hr is Time4 h, which simply referred to as 4 h
The 4^{th} hr in serum is Time 4 h (serum)
PF127-[A] is PF127-pDMAEMA
PF127-[B] is PF127-p(DMAEMA-tBA)
PF127-[C] is PF127-p(DMAEMA-AA)

When a concentration of the Pluronic® is higher than CMC, it will self-assemble into a micelle. The micelle is prepared by emulsion in the present invention. Firstly, the drug and the polymer carrier are dissolved in THF and stably dispersed in water. The polymer carrier forms the micelle, wherein the hydrophobic block and the drug are in the core of the micelle and the hydrophilic block outwardly forms the shell part.

The CMC, the particle size distribution and the zeta potential of the carrier are observed, and then the encapsulation efficiency and the regulation for releasing the drug under various pH values of the carrier are investigated after encapsulating an anticancer drug, EPI. As shown in Table 11, at pH=7.4, the free EPI is quickly released in a short time, and the EPI encapsulated with the carrier can efficiently control the release of the drug; the EPI is released about 80% within 48 hrs. The PP123-b-pDMAEMA/EPI displays a faster releasing phenomenon, and the same release behavior is observed at pH=6.4 and pH=5.6. Because the hydrophobic block of the PL121-b-pDMAEMA/EPI micelle has a larger hydrophobic core and forms stronger hydrophobic force with the drug, its drug releasing rate is lower. PF127-b-pDMAEMA has a longer hydrophilic block length which goes around the outside of the hydrophobic drug and causes the difficulty in drug releasing. In contract, PP123-b-pDMAEMA/EPI having a close hydrophilic and lipophilic ratio shows a fastest release rate. When the hydrophilic ratio is larger than the lipophilc ratio, the hydrophilic block is at the outside of the inner core and increases the steric barrier, which causes difficulty in releasing the drug. When the lipophilic and hydrophilic ratio is close, the drug releasing environment exhibits a better condition and the drug releasing rate is the fastest. Because EPI neutralizes the positive charge of pDMAEMA, the sensitivity of pDMAEMA to a pH change is insignificant. This situation leads to no significant impact on the EPI release rate of the three block copolymers in the various pH values

TABLE 11
Analysis of Drug Release Rates

| | | PF127-[A]/ | PP123-[B]/ | PL121-[C]/ |
|---|---|---|---|---|
| | | EPI | EPI | EPI |
| Drug releasing | | | | |
| time at pH = 7.4 | | | | |
| 0 | h | 0(%) | 0(%) | 0(%) |
| 0.5 | h | 13.6 ± 2.4(%) | 14.3 ± 1.1(%) | 15.7 ± 1.1(%) |
| 1 | h | 23.2 ± 2.2(%) | 24.2 ± 1.9(%) | 25.6 ± 0.7(%) |
| 2 | h | 32.2 ± 2.4(%) | 36.5 ± 2.2(%) | 35.1 ± 0.9(%) |
| 4 | h | 42.1 ± 4.1(%) | 48.6 ± 2.6(%) | 44.6 ± 1.3(%) |
| 8 | h | 52.5 ± 4.6(%) | 59.6 ± 3.1(%) | 53.9 ± 2.0(%) |
| 12 | h | 61.5 ± 5.5(%) | 68.4 ± 2.6(%) | 61.6 ± 2.1(%) |
| 24 | h | 72.0 ± 2.2(%) | 61.5 ± 5.5(%) | 71.2 ± 1.9(%) |
| 48 | h | 84.0 ± 0.9(%) | 91.7 ± 0.4(%) | 83.4 ± 0.6(%) |
| Drug releasing | | | | |
| time at pH = 6.4 | | | | |
| 0 | h | 0(%) | 0(%) | 0(%) |
| 0.5 | h | 24.2 ± 5.7(%) | 21.4 ± 6.7(%) | 29.5 ± 2.5(%) |
| 1 | h | 32.4 ± 6.1(%) | 34.7 ± 4.3(%) | 38.5 ± 2.6(%) |
| 2 | h | 42.2 ± 5.8(%) | 48.2 ± 0.9(%) | 46.6 ± 2.2(%) |
| 4 | h | 50.7 ± 4.9(%) | 57.7 ± 2.6(%) | 53.4 ± 2.0(%) |
| 8 | h | 58.6 ± 4.5(%) | 64.6 ± 6.2(%) | 59.7 ± 3.0(%) |
| 12 | h | 65.1 ± 3.3(%) | 70.4 ± 6.8(%) | 65.0 ± 2.6(%) |
| 24 | h | 73.5 ± 2.2(%) | 80.7 ± 8.2(%) | 73.6 ± 1.7(%) |
| 48 | h | 88.8 ± 2.9(%) | 89.9 ± 3.7(%) | 86.8 ± 5.5(%) |
| Drug releasing | | | | |
| time at pH = 5.6 | | | | |
| 0.5 | h | 10.9 ± 1.3(%) | 16.9 ± 1.2(%) | 11.9 ± 1.4(%) |
| 1 | h | 16.9 ± 2.9(%) | 28.8 ± 1.1(%) | 19.9 ± 0.4(%) |
| 2 | h | 30.5 ± 1.1(%) | 39.3 ± 0.7(%) | 29.7 ± 1.9(%) |
| 4 | h | 40.2 ± 0.2(%) | 49.9 ± 0.4(%) | 39.9 ± 0.9(%) |
| 8 | h | 51.5 ± 3.0(%) | 60.2 ± 0.5(%) | 51.7 ± 2.8(%) |
| 12 | h | 59.5 ± 1.8(%) | 70.4 ± 0.4(%) | 60.1 ± 3.1(%) |
| 24 | h | 69.3 ± 0.2(%) | 78.6 ± 0.2(%) | 68.7 ± 3.6(%) |
| 48 | h | 84.9 ± 8.6(%) | 88.2 ± 1.8(%) | 79.2 ± 4.7(%) |

Note:
EPI is Epirubicin
PF127-[A]/EPI is PF127-pDMAEMA/EPI
PP123-[B]/EPI is PP123-pDMAEMA/EPI
PL121-[C]/EPI is PL121-pDMAEMA/EPI

Human oral cancer cell line (KB cell line) is used to investigate the cell viability of the carrier and the drug-loaded micelle, and the expression of the drug-loaded micelle through the endocytosis is observed using a confocal laser scanning microscope (CLSM). The 4′,6-diamidino-2-phenylindole (DAPI) and the lysosomal probe (Lyso-Traker) are used for cell core staining and the lysosome staining, respectively. The drug at the concentration of 0.625 μg/mL is added according to the cell viability for the drug-loaded micelle. The KB cell line is cultured for 0.5, 1, 3, and 24 hrs, in which the blue parts are shown by the DAPI staining at the cell core; the red is the fluorescence of the EPI; the green is the fluorescence of the Lyso-Traker, and the purple is the overlap of EPI and DAPI and the yellow is the overlap of Lyso-Traker and EPI. The amounts of EPI entering the nuclei and the lysosomes (the purple and the yellow images) increase with the increase in the incubation time.

Free EPI fluorescence is observed in the cell nuclei rather than in cytoplasm when cells are incubated for 1 h. After the drug is encapsulated with the micelle, the entering routes of the micelle and free drug may be different. Compared the images of three drug-loaded micelles with that of EPI, the color in purple due to the overlapping of DAPI and EPI becomes clearer at a longer incubation time. This result demonstrates that the encapsulated EPI takes more time than free EPI to enter the cell nuclei.

In Vitro Cell Viability Test:

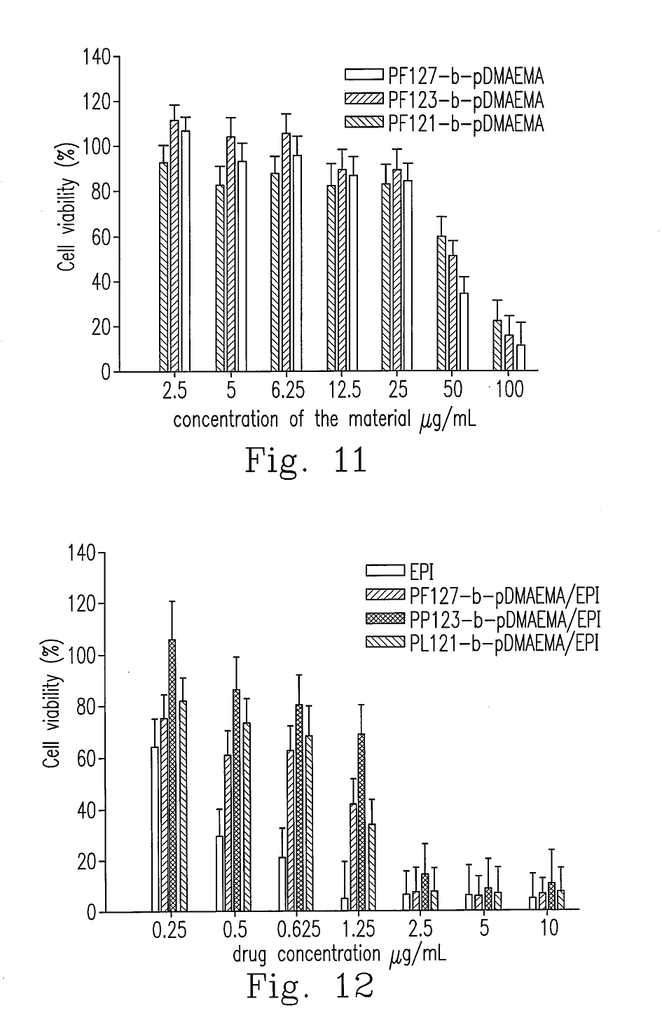

An MTT assay is used to determine cytotoxicity. In FIG. 11, the cell viability of each group of the {PPEO}-pDMAEMA block polymers may substantially achieve 80% when the concentration is within 2.5˜25 μg/mL. Along with increasing the concentration, the cell viabilities of PP123-b-pDMAEMA and PL121-b-pDMAEMA decrease, especially in the PL121-b-pDMAEMA.

The Cytotoxic Test (IC₅₀) for the EPI-Loaded Micelles

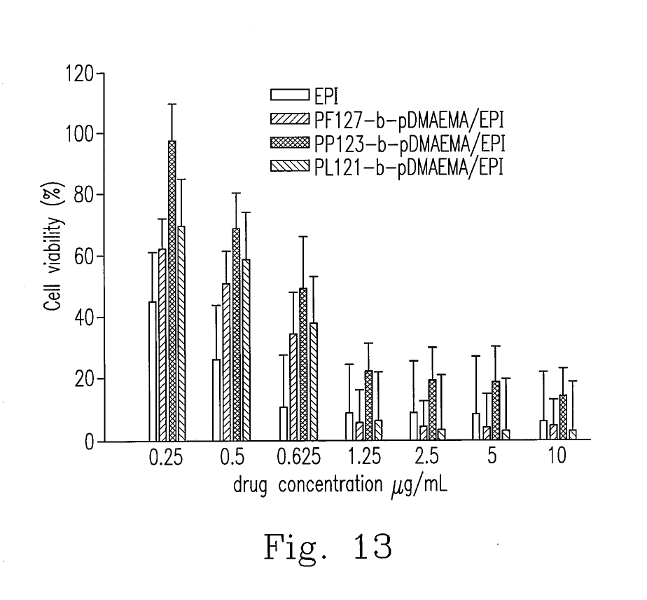

Since the EPI exhibits its cell-killing effect after conjugating with the DNA in the cell core, Table 12 shows the half maximal inhibitory concentration (IC₅₀) for the drug and the loaded micelles. In FIG. 12, the cytotoxicity of EPI on the cancer cells decreases in the EPI-encapsulated micelle for 24 hrs. As shown in FIG. 13, the cytotoxicity enhances for 48 hrs because of increasing the reaction time of the drug and DNA. The releasing rate of the drug from the carrier and the action time of the drug and DNA are both factors on cytotoxicity. The EPI-loaded PP123-b-pDMAEMA micelle has the highest IC₅₀ value.

TABLE 12
IC₅₀ values of the drug and the micelles in cell culture

Incubating time

| Micelles | 24 hr | 48 hr |
|---|---|---|
| EPI | 0.35 μg/mL | 0.20 μg/mL |
| PF127-[A]/EPI | 1.00 μg/mL | 0.45 μg/mL |
| PP123-[A]/EPI | 1.58 μg/mL | 0.79 μg/mL |
| PL121-[A]/EPI | 0.94 μg/mL | 0.55 μg/mL |

Note:
EPI is Epirubicin
PF127-[A]/EPI is PF127-b-pDMAEMA/EPI
PP123-[A]/EPI is PP123-b-pDMAEMA/EPl
PL121-[A]/EPI is PL121-b-pDMAEMA/EPl

The present invention prepares PF127-pDMAEMA block copolymer of three different pDMAEMA block lengths using the atom transfer radical polymerization (ATRP), and investigates gene expressions of the different pDMAEMA block lengths by an in vitro test. The result shows that the high pDMAEMA block length has an excellent gene transferring ability but has a high cytotoxicity as well. The cell damage caused by the high molecular weight pDMAEMA block cannot be reduced even when a high biocompatible Pluronic is introduced. However, the introduction of the active functional group (—COOH) decreases the cytotoxicity, and remains a high gene transfection efficiency as well. Moreover, this active COOH functional group may be used to react with an active ligand, which targets tumor tissue, or serves as a real-time tracking agent for molecular imaging. In summary, the optimal synthetic condition and length are chosen for preparing an optimal pDMAEMA block having excellent gene transfection efficiency and decreasing cytotoxicity as a non-viral gene carrier. The introduced functional molecule may be further manipulated to prepare a nanoparticle of the multifunctional modality. This nanoparticle can carry a gene/anticancer drug and preferably targets the cancer cells as a multifunctional reagent for the cancer therapy and diagnosis.

The Biological Experiment

Materials:
Luria-Bertani agar plates:
   Ampicillin 100 mg/mL
   Kanamycin 25 mg/mL
   Isopropyl-β-D-1-thiogalacto-pyranoside (IPTG) 1 M
   Protoplasting buffer is prepared by 15 mM Tris-HCl at pH 8.0, 0.45 M sucrose and 8 mM EDTA and stored at 4° C.
   Lysozyme 50 mg/mL
   Gram-negative lysing buffer is prepared by 10 mM Tris-HCl at pH 8.0, 10 mM NaCl, 1 mM sodium citrate and 1.5% sodium dodecyl sulfate (SDS).
   diethylpyrocarbonate (DEPC)
   The saturated NaCl solution is prepared by mixing the 40 g of NaCl dissolved in 100 mL of DEPC and the dH₂O.
   Absolute alcohol and 70% alcohol
RPMI 1640 medium
   10% fetal bovine serum (FBS), Streptomycin 100 μg/mL, Penicillin 100 U/mL, L-glutamine 2 mM and 90% sodium bicarbonate (NaHCO₃) 1.5 g/L.
DMEM medium (Dubecco's Modified Eagle Medium)
   10% fetal bovine serum (FBS), 20 ml of glutamine, 20 ml of antibiotics, 3 g of NaHCO₃, DMEM powder, 2.38 g of 2-[4-(2-Hydroxyethyl)-1-piperazinyl]ethane sulfonic acid (HEPES) dissolved in 2 L of ddH2O, and the pH value is adjusted to 7.2.
Phosphate buffered saline (PBS) buffer: 0.27 g of KH₂PO₄, 1.42 g of Na₂HPO₄.12H₂O, 8 g of NaCl and 0.2 g of KCl are dissolved in the distilled water (about 800 mL) by completely stirring, the pH value is adjusted to 7.4 by the conc. HCl to form a 1 L solution. After the solution is sterilized under high temperature and high pressure, it is stored at room temperature.
TAE buffer is prepared as a pH 8.0 electrophoresis buffer by 40 mM
   Tris(hydroxymethyl)amino-methane acetate salt (Tris acetate) and 1 mM ethylenediaminetetraacetic acid (EDTA).
Cell lysis buffer: 10 ml of mammalian protein extraction reagent (T-PER™) is precolded thereby a tablet of the complete mini protease inhibitor cocktailis added for mixing evenly. The mixture is stored at −80° C.
Epirubicin.HCl is dissolved in a small amount of the ddH₂O to form a preparation.

The Analysis for the Structure, Transforming Rate and the Degree of the Polymerization of the Five-Blocks Copolymer

The Pluronic® modification and the analysis for the structure, transforming rate and the degree of the polymerization of the five-blocks copolymer are performed using the Fourier Transform Infrared Spectroscope (FT-IR) at wavelength 4000 cm⁻¹˜400 cm⁻¹ to observe the functional group transformation of the copolymer for ensuring the reaction state. ¹H-NMR is used for structural analysis. The block copolymer is completely dissolved in the THF at a concentration of 1 mg/mL. The weight-average molecular weight (M_{w}) and the number-average molecular weight (Mₙ) of block copolymers are determined by injecting 10 μL of sample into the gel permeation chromatography (GPC) using THF as a mobile phase. The molecular weight distribution index (M_{w}/Mₙ) of the block polymers is also calculated.

The repeating unit DMAEMA in the block copolymers is calculated from the ¹H-NMR integral ratio and presents as the degree of polymerization (DP). The DP of the PL121-b-pDMAEMA, PP123-b-pDMAEMA and PF127-b-pDMAEMA are 33, 34 and 38, respectively.

DP of the Pluronic®-b-pDMAEMA

DP=[(integral_{2.26 ppm}+integral_{2.70 ppm})/6]÷[(integral_{1.15 ppm}/PPO repeating unit×3)]

poly propylene oxide (PPO)

The Micelle Experiment

Critical Micelle Concentration (CMC)

Three stock solutions are diluted in distilled water to make concentrations ranging from 1 to 2×10⁻³ (mg/mL), respectively. Pyrene is used as a fluorescent probe, which is dissolved in acetone at a concentration 6.0×10⁻⁷ M. The fluorescence intensity ratio at 339 nm and 334 nm is measured by fluorescence spectroscopy. The excitation and emission wavelengths of the pyrene are set at 330 nm and 390 nm.

Micelle Preparation

Five mg of a block copolymer is dissolved in 500 μL of THF. The solution was then added dropwise to 5 mL of distilled water under the sonication for 3 min. The THF is removed under the reduced pressure and the solid micelle is obtained after freeze-drying. The ratio of the above-mentioned distilled water and the THF is 10:1 (v/v). The concentration of micelles dispersing in water is 1 mg/mL.

Particle Size and Zeta Potential of Micelles

One mg of micelle carrier is dispersed in 1 mL of distilled water, and the particle size and zeta potential of the micelle carrier are measured by DLS. The measured particle sizes range from 1 to 5000 nm; the temperature is 25° C., and the scattering light angle is 90°.

Analysis for the Morphology of Micelles

One mg of the micelle carrier prepared from each block copolymer is dispersed in 1 mL of distilled water and dropped on the carbon coated copper grid. After the micelle has been dried, the morphology and particle size of the micelle is observed by TEM.

In Vitro Experiment of Micelles

Cytotoxic Test for the Micelle Carrier

KB cells (human oral carcinoma cell line) are seeded in 96-well plate at 5×10³ cells/well under 5% CO₂, 37° C. in 100 μL of RPMI 1640 medium for 24 hrs.

On the second day, the respective 100 μL/well medium containing the micelle carrier (200, 100, 50, 25, 12.5, 6.25 and 5 μg/mL) is added into the plate for another 24 hr-incubation.

50 μL of 2 mg/mL MTT is added into each well of the plate. After 3 hrs, the supernatant is removed by 1500 rpm centrifugation for 15 min. 100 μL/well dimethyl sulfoxide (DMSO) is added into the plate for evenly vibrating over 20 min and the 595 nm absorption (OD₅₉₅) is measured in an ELISA reader. The formula for calculating the cell viability is:

Cell viability(%)=(OD_{595(experimental group)}/OD_{595(control group)})×100.

Construction of the Gene Carrier

Plasmid DNA Preparation

The nucleic acids of the plasmid DNA carrying the green fluorescent protein gene (pEGFP-Cl) and the control group (pGL3-Control) contain the Kanamycin and Ampicilin drug resistant gene sequence respectively.

The plasmid nucleic acid DNA carrying green fluorescent protein gene (pEGFP-Cl, 4.7 kb)

This plasmid DNA contains a reporter gene sequence of the enhanced green fluorescent protein (EGFP), the transcribed and translated protein can express the green fluorecin (excitation at 488 nm and emission at 507 nm). The green fluorescent protein absorbs the blue exciting light through the cyclo-structure of the residue of the amino acid such as serine (Ser), tyrosine (Tyr) and glycine (Gly), to generate the green fluorecin. Further, the plasmid DNA has a cytomegalovirus promoter (CMV promoter), and the fluorecin expressed in the human cell is observed using the fluorescent microscope after being excited without any assistant enzyme or substrate addition.

The Plasmid DNA Control Group (pGL3-Control, 5.3 kb)

The plasmid DNA has the reporter gene sequences of the luciferase (luc⁺), Simian vacuolating virus 40 promoter (SV40 promoter) and enhancer. It generates the luciferase via transcription and translation. The luciferase catalyzes the luciferin to perform oxidation to generate a luciferous protein. Thus, the pGL3-Control preferably provides the bases for detecting the transfection efficiency and quantitative analysis.

Plasmid DNA Extraction

The pEGFP-Cl plasmid DNA is firstly transformated into Escherichia coli, the trasformated DH5α is incubated in LB broth containing 1 mM kanamycin for 16˜18 hrs (37° C. at 200 rpm). After being incubated, the plasmid DNA is extracted according to the extracting steps of the Maxi-V500™ Ultrapure Plasmid Extraction System Kit (Viogene). When the concentration of the purified double strand DNA solution is 50 μg/mL, the OD₂₆₀ value is 1.0 and OD₂₆₀/OD₂₈₀ ratio is 1.8. However, the OD₂₆₀/OD₂₈₀ ratio is lower than 1.8 while the DNA is contaminated by the protein. Therefore, the purity and concentration of the plasmid DNA are determined by NanoDrop 1000 (Thermo Fisher Scientific) and the DNA is stored at −20° C.

Preparation and Analysis of the Block Copolymer/DNA Polyplex

Preparing method: Plasmid DNA (pEGFP-Cl or pGL3-Control) is formulated at 1 mg/mL in ddH₂O. Different kinds of the PF127-pDMAEMA-L, PF127-pDMAEMA-M, PF127-pDMAEMA-H, PF127-pDMAEMA, PF127-p(DMAEMA-tBA) or PF127-p(DMAEMA-AA) block copolymers are dissolved in the ddH₂O and controlled at a concentration of 2 mg/mL.

Each block copolymer and DNA are mixed through vortex, and the vortex is continued over 1 min for preparing the polyplexes with different N/P ratios (1, 3, 6, 9 and 12). The polyplexes are incubated 30 min for experiments.

The Electrophoresis of the Block Copolymer/DNA Polyplex:

0.8% Agarose gel solution containing 1 μg/mL ethidium bromide (EtBr) is prepared; it is heated, poured into the model and cooled to form a gel. An appropriate amount of 1×TAE buffer is poured into the horizontal electrophoresis, and the prepared block copolymer/DNA polyplexes having various N/P ratios are placed into the holes on the gel for 40 min electrophoresis (100 V). After the electrophoresis, the result is captured and recorded by irradiated the gel with the UV light (λ=365 nm) in the 2UV Transilluminator within the dark box of the gel imaging system.

Particle Size Distribution and Zeta Potential Determination of the Block Copolymer/DNA Polyplex

Particle size distribution and zeta potential of the block copolymer/DNA polyplex are measured using Malvern Zatasizer (Malvern Instrument, England). The range of the measured particle size is set between 1˜5000 nm; the temperature is 25° C. and the emission light angle is 90°. The zeta potential of the polyplex is detected using Aqueous Dip Cell under the automatic model.

Morphological Analysis of the Block Copolymer/DNA Polyplex:

Ten μL of prepared polyplex is dropped on the carbon coated copper grid, which is placed in a plastic box covered with aluminum foil with pin-size holes and be placed at 37° C. in an oven for 3˜5 days. After the polyplex is dry, it is analyzed by the transmission electron microscopy (TEM).

Cytotoxic Test for the Drug-Loaded Micelle:

Drug-Loaded Micelle Preparation:

The salt of the Epirubicin.HCl (EPI.HCl) is removed using triethylamine (TEA) and dissolved in THF. The half mg EPI.HCl is added into the THF solution at a concentration of 1 mg/mL, and TEA is dropwise added into the solution using the molar ratio EPI.HCl:TEA=1:3. After sonication for 30 min, the mixture is diluted to five concentrations, 1.25×10⁻², 6.25×10⁻³, 3.125×10⁻³, 1.5625×10⁻³ and 7.8125×10⁻⁴ mg/mL, respectively. The fluorescent intensity at 591 nm is obtained using a fluorescence spectrometer and a diagram is plotted with respect to the fluorescent intensity vs. concentration to create a calibration curve. The excitation wavelength of EPI is 470 nm.

Five mg of block copolymer is dissolved in 500 μL of drug-containing THF, and the solution was then added dropwise to 5 mL of distilled water and under the sonication for 3 min. The THF is removed under the reduced pressure; the unloaded EPI is filtered out using 0.45 μm filter, and the solid is obtained via the freeze drying. The ratio of the above-mentioned distilled water and the THF is 10:1 (v/v). The concentration of micelles dispersing in water is 1 mg/mL.

Encapsulating Ratio Test:

The drug is extracted out from drug-loaded micelles by THF. The fluorescent intensity of the drug is measured using the fluorescence spectrometer and the amount of the encapsulated drug is obtained based on the calibration curve.

Drug Encapsulating Ratio Calculation:

The drug encapsulation efficiency (EE %) is the ratio of the amount of drug in micelle and the amount of drug in feed, while the drug loading efficiency (LE %) is the ratio of the amount of drug in micelle and the sum of the amount of polymer and the amount of drug in micelle.

EE(%)=(amount of drug in micelle/amount of drug in feed)×100

LE(%)={amount of drug in micelle/(amount of polymer+amount of drug in micelle)}×100

Drug Release

Epirubicin UV Absorption Calibration Curve

Epirubicin.HCl is dissolved in PBS buffer at pH=7.4, 6.5 and 5.5, respectively. TEA is dropwise added into the solution using the molar ratio EPI.HCl:TEA=1:3 to remove the salt, and the mixture is formulated to five concentrations. The 480 nm UV absorption value is obtained and a diagram is plotted with respect to the absorption value vs. concentration for regression.

Drug Release:

1.6 mg micelle containing EPI is dispersed in 10 mL PBS buffer at pH 7.4, 6.5 and 5.5, which is placed in a dialysis membrane (cut-off molecular weight 3500, length=5 cm, width=2.9 cm) and immersed in 30 mL PBS buffer. The outside PBS buffer is collected at different time points and then the fresh 30 mL PBS buffer is replaced. The drug concentration is measured and calculated based on a calibration curve generated in PBS buffer with the same pH value.

The results of the cytotoxic test for the drug-loaded micelle are presented as the half maximal inhibitory concentration, i.e. IC₅₀, which is used to examine the biological or biochemical inhibitory effect of a compound. Thus, the IC₅₀ represents the concentration of the drug-loaded micelle required to inhibit the growth of a half of the cancer cells.

5×10³ cells/well KB cells are incubated in 100 μL of RPMI 1640 medium, and transferred into the 96-well plate for incubating 24 hrs.

Epirubicin.HCl and drug-loaded micelle is prepared at 20 μg/mL, in RPMI 1640 medium. After a serial of dilution, 100 μL/well of each concentration (20, 10, 5, 2.5, 1.25, 1 and 0.5 μg/mL) is added into the cultured plate for 24 hrs incubation.

After 24 hrs incubation, the medium containing the drug and the micelle is removed, and the plate is washed with 100 μL/well PBS buffer at pH=7.4 and further incubated in 100 μL/well fresh medium for 24 and 48 hrs.

The plate is added with 50 μL/well (2 mg/mL) MTT and placed in the incubator for 3 hrs. After centrifugation at 1500 rpm for 15 min, the supernatant is removed and 100 μL/well DMSO is added into the plate for evenly shaking over 20 min. Then, the cell viability is calculated using the ELISA reader.

Intracellular delivery is observed using the confocal microscope. 18 mm cover slip is immersed in 0.1N HCl solution for one day and washed to remove the impurities on the slide. After being rinsed by the ddH₂O, the slide is wiped and then immersed in 75% alcohol. In the laminar flow, the slide is taken out by a sterilized clip to cause the remaining alcohol to evaporate over the fire, and the slide is placed in a 12-well plate.

1×10⁵ cells/well KB cells are cultured in 1 mL RPMI 1640 medium, and the cells are transferred to the cultured plate containing the slides for 24 hrs. 1 mL of the medium containing 1 μM Lyso-Tracker Red probe is added into the plate. After 30 min, the medium is removed and the plate is washed with PBS and refilled with 1 mL fresh medium.

Epirubicin.HCl and the drug-loaded micelle are prepared as 0.625 μg/mL in the cultured medium, and the medium is added into the cultured plate containing the slides with the cells.

After 0.5, 1, 3 and 24 hrs, the medium containing the drug is removed and the slides containing the cells are removed and washed with PBS buffer at pH=7.4 for three times. The slides are put in a new plate and 1 mL/well of 3.7% pareformaldehyde is added to the plate, which is placed in a 37° C. incubator for 30 min to fix the cells on the slides. Subsequently, the pareformaldehyde is removed and the plate is washed with pH=7.4 PBS buffer for three times.

The plate is added with 1 mL/well of 0.1% mono(p-(1,1,3,3-tetramethylbutyl)phenyl)ether (Triton) X-100 and placed in a 37° C. incubator for 5 min. Subsequently, the Triton X-100 is removed and the plate is washed with pH=7.4 PBS buffer for three times.

0.5 mL/well of 0.5 μg/mL (2-(4-Amidinophenyl)-6-indolecarbamidine dihydrochloride (DAPI) is added into the plate, and the plate is incubated in a 37° C. incubator for 5 min to perform the cell nuclei staining. Subsequently, the DAPI is removed and the plate is washed with PBS buffer at pH=7.4 for three times.

One drop of the fluorescent mounting medium is dropped on the slide, and the cover slip is placed cell side down on the mounting medium and then sealed by nail polish around the edges. When the nail polish is dry, the slide can be observed by CLSM.

Cytotoxic Test for the Polyplex:

1×10⁵ human embryonic kidney 293T (HEK 293T) cells are seeded in each well of a 12 well plate in 1 ml/well DMEM medium containing 10% FBS for 24 hrs.

On the second day, the culturing medium is replaced by 1 mL/well serum-free DMEM medium, and then the polyplexes formed by various N/P ratios with 1 μg DNA are placed in the medium. After 4 hrs incubation, the medium is replaced by the fresh DMEM medium containing 10% FBS.

After 68 hrs incubation, 1 mL of 5 mg/mL MTT reagent is added into each well of the plate and the plate is placed in an incubator for 3 hrs to enable the formazan crystal to pierce the cell membrane. After 3 hrs, the cells are collected and transferred to a 15 mL centrifuge tube and centrifuged at 1000 rpm for 15 min. The supernatant is carefully drawn out and 1 mL of DMSO is added in an eppendorf to dissolve the formazan crystal, and the eppendorf is evenly shaken for 15 min.

100 μL/well of the above crystal solution dissolved in the DMSO is added into a 96-well plate, and the 595 nm absorption is read using the ELISA Reader. The obtained values are put into an equation for calculating the cell viability.

Gene Transfection Efficiency:

The gene transfection efficiency is quantitated using the relative luciferase unit (RLU) relative to the pGL3-Control.

1.0×10⁵/well of the HEK 293T is seeded in each well of the 12-well plate in 1 mL/well DMEM medium containing 10% FBS for 24 hrs.

On the second day, the medium is replaced by 1 mL/well DMEM medium (containing with or without 10% FBS), and the polyplexes formed by various N/P ratios with 1 μg DNA are placed in the plate. After 4 hrs, the medium is replaced with the fresh DMEM medium containing 10% FBS.

After 68 hrs, the medium is collected and added with 200 μL/well cell lysis buffer. The cells are incubated at a −20° C. refrigerator overnight to cause the ice crystal to pierce the cell membrane.

On the second day, different concentrations of the bovine serum albumin (BSA) standards are prepared according to the method provided by the BCA Protein Assay Kit. The buffer A and buffer B are mixed in a ratio of 50:1 to make a working reagent (WR).

The cells stored overnight at the −20° C. refrigerator are warmed to the room temperature, and the cells and the cell lysis buffer are extracted and transferred to an eppendorf and centrifuged at 15000 rpm for 30 min.

The supernatant A is extracted after centrifugation, and the supernatant (50 μL/well, 3 wells) is added into a 96 well plate. The RLU value is measured using TopCount NXT™ (Perkin Elmer).

10 μL/well of BCA standard solution and the supernatant A are loaded into the 96-well plate, respectively. 200 μL/well WR is added for evenly shaking over 15 min. The 96-well plate is protected from light and placed in the incubator. After 30 min, the plate is cooled to the room temperature and then the 595 nm absorption is read using the ELISA Plate Reader (E-Lab).

The value measured by the ELISA reader is put into a calibration curve to calculate the amount of the luciferin protein (mg). The RLU over the amount of the protein (mg) is plotted to illustrate the gene expression.

### Embodiment 1

### PF127-p(DMAEMA) Synthesis

Preparation of the Bromo Modifier of Pluronic® F127

12.6 g (1 mmol) Pluronic® F127 (PF127, M_{w}=12600 g/mol) is placed in a double-necked flask to degas under vacuum for 30 min, and 20 mL dichloromethane is filled in under argon. While the reactants are completely dissolved, 0.7 mL (5 mmol) triethylamine is added into the solution under ice bath. After stirring for about 15 min, 0.6 mL (5 mmol) 2-bromoisobutyryl bromide is then added into the solution. The reaction is carried out for 48 hrs at room temperature.

The reaction product is rinsed with large amount of hexane to remove the un-reacted 2-bromoisobutyryl bromide and incubated in a 4° C. refrigerator. The supernatant is poured out; a precipitate is obtained by repeated washing the product with hexane, and the precipitate is then extracted with 0.4 M HCl solution for many times to remove the salts produced in the reaction process. Finally, the purified bromo modifier of Pluronic® F127 (PF127-Br) is dried under vacuum for 2 days. PF127-Br is dissolved in CDCl₃ and ¹H-NMR spectrum is acquired to confirm the chemical structure and the degree of bromination.

Preparation of the Pentablock Copolymer Pluronic®F127-block-poly(2-(dimethylamino)ethyl methacrylate (PF127-p(DMAEMA))

250 mg (0.02 mmol) PF127-Br is placed into a double-necked flask and degassed for 30 min and filled in the argon atmosphere. Subsequently, 0.4 mL distilled water and 1.6 mL 2-propanol are added into the flask to completely dissolve the PF127-Br. 0.16 mL (1 mmol) 2-(dimethylamino)ethyl methacrylate (DMAEMA) is then added into the flask. After the solution mixture is repeatedly frozen and thawed for 6 times to remove oxygen, 5.6 mg (0.04 mmol) CuBr and 6.1 mg (0.04 mmol) 2,2′-bipyridine (Bpy) are added under argon atmosphere. The polymerization was done at room temperature for 2 hrs.

The product of the polymerization passes through the cut-off (M_{w}=3500) to remove the un-polymerized monomer. After the frozen-dried, the product is dissolved in toluene; the cationic exchange resin Amberlite® IR120 is used to remove the 2,2′-bipyridine. CuBr is removed via the Al₂O₃-filled column. The product is precipitated in hexane and centrifuged. The supernatant is poured out and the product is dried under vacuum to yield the PF127-p(DMAEMA) copolymer. The product is dissolved with D₂O, and ¹H-NMR is measured for determining the chemical structure and the monomer conversion.

### Embodiment 2

### PL121-p(DMAEMA) Synthesis

Preparation of the Bromo Modifier of Pluronic® L121

4.40 g (1 mmol) Pluronic® L121 (PF121, M_{w}=4400 g/mol) dissolved in 20 mL dichloromethane is placed in a double-necked flask and degassed for 30 min and filled in an argon atmosphere. While the reactants are completely dissolved, 0.7 mL (5 mmol) triethylamine is added into the solution under ice bath. After stirring for about 15 min, 0.6 mL (5 mmol) 2-bromoisobutyryl bromide is then added into the solution and the reaction is carried out for 48 hrs at room temperature.

The reaction product is rinsed with large amount of hexane to remove the un-reacted 2-bromoisobutyryl bromide and incubated in a 4° C. refrigerator. The supernatant is poured out; the precipitate is obtained by repeated washing the product with hexane, and the precipitate is then extracted with 0.4 M HCl solution several times to remove the salts produced in the reaction process. Finally, the purified bromo modifier of Pluronic® L121 (PL121-Br) is dried under vacuum for 2 days. PL121-Br is dissolved in CDCl₃ and ¹H-NMR spectrum is acquired to confirm the chemical structure and the degree of bromination.

Preparation of the Pentablock Copolymer Pluronic®L121-block-poly(2-(dimethylamino)ethyl methacrylate (PL121-p(DMAEMA))

91 mg (0.02 mmol) PL121-Br is placed in a double-necked flask and degassed for 30 min and filled in the argon atmosphere. Subsequently, 0.4 mL distilled water and 1.6 mL 2-propanol are filled into the flask to completely dissolve the PL121-Br. 0.16 mL (1 mmol) 2-(dimethylamino)ethyl methacrylate (DMAEMA) is then added into the flask. After the solution mixture is repeatedly frozen and thawed for 6 times to remove oxygen, 5.6 mg (0.04 mmol) CuBr and 6.1 mg (0.04 mmol) 2,2′-bipyridine (Bpy) are added under argon atmosphere. The polymerization was done at room temperature for 2 hrs. The product of the polymerization passes through the cut-off (M_{w}=3500) to remove the un-polymerized monomer. The solid obtain by lyophilization is re-dissolved in toluene, the cationic exchange resin Amberlite®IR120 is used to remove the 2,2′-bipyridine. CuBr is removed via the Al₂O₃-filled column. The product is precipitated in hexane and centrifuged. The supernatant is poured out and the product is dried under vacuum to yield the PL121-p(DMAEMA) copolymer. The product is dissolved with D₂O, and ¹H-NMR is measured for determining the chemical structure and the monomer conversion.

### Embodiment 3

### PP123-p(DMAEMA) Synthesis

Preparation of the Bromo Modifier of Pluronic® P123:

5.80 g (1 mmol) Pluronic® P123 (PP123, M_{w}=5600 g/mol) dissolved in 20 mL dichloromethane is placed in a double-necked flask and degassed for 30 min and filled in an argon atmosphere. While the reactants are completely dissolved; the purified bromo modifier of Pluronic® P123 (PP123-Br) is obtained according to the above embodiments. The ¹H-NMR spectrum of PP123-Br is measured.

Accordingly, 118 mg (0.02 mmol) PP123-Br is placed in a double-necked flask and degassed for 30 min and filled in the argon atmosphere. The pentablock copolymer Pluronic® P123-block-poly(2-(dimethylamino)ethyl methacrylate (PP123-p(DMAEMA)) is obtained according to the above embodiments. ¹H-NMR spectrum thereof is measured.

### Embodiment 4

### Preparation of the Block Copolymer PF127-p(DMAEMA-Acrylic Acid) (PF127-p(DMAEMA-AA))

250 mg (0.02 mmol) bromo modifier of Pluronic® F127 (PF127-Br) is placed in a double-necked flask and degassed for 30 min and filled in the argon atmosphere. Subsequently, 0.4 mL distilled water and 1.6 mL 2-propanol are filled into the flask to completely dissolve the PF127-Br. 0.6 mL (4 mmol) DMAEMA monomer and 0.1 mL (1 mmol) tert-butyl acrylate (tBA) monomer are then added into the flask. After the solution mixture is repeatedly frozen and thawed for 6 times to remove oxygen, 5.6 mg (0.04 mmol) CuBr and 6.1 mg (0.04 mmol) 2,2′-bipyridine (Bpy) are added under argon atmosphere. The polymerization was done at room temperature for 2 hrs. The product of the polymerization passes through the cut-off (M_{w}=3500) to remove the un-polymerized monomer. After lyophilization, the product is dissolved in toluene, the cationic exchange resin Amberlite®IR120 is used to remove the 2,2′-bipyridine. CuBr is removed via the Al₂O₃-filled column. The product is precipitated in hexane and centrifuged. The supernatant is poured out and the product is dried under vacuum to yield the PF127-p(DMAEMA-tert-Butyl acrylate) (PF127-p(DMAEMA-tBA) copolymer. The product is dissolved with D₂O, and ¹H-NMR is measured for determining the chemical structure and the monomer conversion.

100 mg (0.05 mmol) block copolymer PF127-p(DMAEMA-tBA) dissolved in 10 mL disₜiₗled water is placed into a double-necked flask followed by adding 0.2 mL of 0.1N HCl into the solutᵢon. The reaction is carried out at 40° C. for 24 hrs. HCl is removed using a dialysis membrane (cut-off molecular weight 3500), and the block copolymer PF127-p(DMAEMA-Acrylic acid) is obtained after freeze-dried. The block copolymer PF127-p(DMAEMA-Acrylic acid) is dissolved with D₂O and ¹H-NMR is used to confirm the chemical structure and the monomer conversion.

### Embodiment 5

### Preparation of the Block Copolymer PF127-p(DMAEMA-Cholic Acid) (PF127-p(DMAEMA-CA)

Preparation of the AMA-CA:

The cholic acid (CA) is completely dissolved in the DMSO.

The double-necked flask is dried and degassed under vacuum for 30 min, 1.5 mL DMSO containing the 146.5 mg 1,1-carbonyldiimidazole (CDI, M_{w}=162.15) is added into the flask, and 2.5 mL DMSO containing the 246 mg colic acid (CA, M_{w}=408.58) is added under argon. After 3 hrs reaction, 1 mL DMSO containing 100 mg 2-aminoethylmethacrylate hydrochloride (AMA, M_{w}=165.62) is added into the double-necked flask. The reaction is carried out for one day and the reaction solvent DMSO is removed by dialysis.

The product is dissolved with dichloromethane; the un-reacted cholic acid is removed by extracting with 10% sodium bicarbonate twice, and the un-reacted AMA is removed by extracting with 10% HCl twice. The final product is dried in a vacuum oven for 2 days.

Preparation of the Block Copolymer PF127-p(DMAEMA-co-AMA-CA):

250 mg PF127-Br and 312 mg AMA-CA are placed into a double-necked flask, which has been dried and degassed under vacuum for 30 min. Subsequently, 2 mL of solvent of a 4:1 volume ratio (methanol:H₂O) is added into the flask. 0.7 mL 2-(dimethylamino)ethyl methacrylate (DMAEMA, M_{w}=157.21) is then added into the flask. After the flask is frozen-dry, it is filled with argon to degas for 6 times, 10 mg 2,2′-bipyridine (Bpy, M_{w}=156.19) and 10 mg CuBr are added under the ice bath for polymerization.

Preparation of the Block Copolymer PF127-p(DMAEMA-co-AMA-CA)

250 mg PF127-Br and 312 mg AMA-CA are placed into a double-necked flask to degas for 30 min by filling the argon in circulation. Subsequently, 2 mL of a 4:1 (methanol:H₂O) methanol solution is filled into the flask to completely dissolve the mixture. 0.7 mL 2-(dimethylamino)ethyl methacrylate (DMAEMA) is then added into the flask. After the solution mixture is repeatedly frozen and thawed for 6 times to remove oxygen, 10 mg 2,2′-bipyridine (Bpy) and 10 mg CuBr are added under an ice bath for polymerization.

The product is purified using a dialysis membrane (cut-off molecular weight 3500) to remove the un-polymerized monomer, and the product is obtained after freeze-drying.

Next, the product is dissolved with a small amount of dichloromethane and precipitated into a large amount of hexane to remove the un-reacted DMAEMA. The supernatant is poured out and the product is placed in a vacuum dry box. The molar ratio of PF127-Br:BPY:CuBrDMAEMA:AMA-CA is 1:1:1:200:30.

### Embodiment 6

### Preparation of the Block Copolymer PF127-p(DMAEMA-Folic Acid) (PF127-p(DMAEMA-FA))

Preparation of the Folic Acid-Poly(Ethylene Glycol) (FA-PEG):

16 g (16 mmole) PEG is dried under reduced pressure to remove moisture. 1.8 g (4 mmole) folic acid is dissolved in 25 mL DMSO followed by adding 0.7 g (4.4 mmole) CDI in a double-necked flask, which has been dried and degassed under vacuum for 30 min before used. The solution is stirred under ice bath for 4 hrs. Subsequently, the dried PEG is added into the solution and the reaction is carried out at room temperature for one day.

The product is washed with the acetone for 5 times, and dried in a vacuum oven.

Preparation of PF127-p(DMAEMA-Folic Acid)

50 mg (0.002 mmol) PF127-p(DMAEMA-AA) is dissolved in distilled water, and the condensation agent, ethyl-dimethyl-amino-propyl carbodiimide (EDAC) (5.8 mg, 0.03 mmol), is added to activate the carboxylate group for 24 hrs. Subsequently, 114 mg (0.03 mmol) FA-PEG is added to the solution and the reaction is carried out at room temperature for 24 hrs. After the reaction, the product is purified using a dialysis membrane (cut-off molecular weight 25K) and the product is obtained via freeze-drying.

### Embodiment 7

### Preparation of the Injection Composition of the DNA Polyplex

The components are taken according to the following amount and dissolved in the liquid for injection applications.

| | | |
|---|---|---|
| PF127-p(DMAEMA-tBA)/DNA | 0.2 | mg/vial |
| liquid for injection (PBS buffer) | 100 | mL |

The liquid for injection (PBS buffer) is sterilized via high temperature and high pressure, and the injection liquid is filled into a vial with PF127-p(DMAEMA-tBA)/DNA (0.2 mg powder). The injection liquid is filtered through the 0.22 μm micropore filter and the vial is sealed for storage.

### Embodiment 8

### Preparation of the Composition of EPI Micelle Capsule

The following components are taken, sieved, and filled into the capsule:

| | | |
|---|---|---|
| PP123-b-pDMAEMA/EPI | 140 | mg |
| Lactose (dilution agent | 8.5 | g |

| | |
|---|---|
| Starch paste (adhesive) | qs |

Lactose and an appropriate amount of starch are taken, grinded in a grinding bowl, and sieved (100 mesh). The sieved PP123-b-pDMAEMA/EPI is mixed with the powder and sieved (20 mesh). The mixture is dried in an oven at 30˜40° C. for 1 hr, and being stirred per 15 min. The water amount in the mixture is determined, and the lubricant, the adhesive and the disintegrating agent are added into the mixture to be filled in the capsules.

### Other Embodiments

1. A {PPEO}-{AFG}-{DV} polymer, wherein
   the {PPEO} is a poly(ethylene glycol)-block-poly(propylene glycol)-block-poly(ethylene glycol) (PEO-PPO-PEO) polymer compound, the {AFG} is a vinyl monomer and the {DV} is an active ligand.
2. A micelle, comprising:
   a poly(ethylene glycol)-block-poly(propylene glycol)-block-poly (ethylene glycol) (PEO-PPO-PEO) polymer compound;
   a vinyl monomer forming a block copolymer with the PEO-PPO-PEO polymer compound; and
   an active ligand conjugating with the block copolymer.
3. A nanoparticle, comprising:
   a poly(ethylene glycol)-block-poly(propylene glycol)-block-poly (ethylene glycol) (PEO-PPO-PEO) polymer compound;
   a vinyl monomer forming a block copolymer with the PEO-PPO-PEO polymer compound; and
   an active ligand conjugating with the block copolymer.
4. A pharmaceutical composition, comprising:
   a pharmaceutical acceptable carrier;
   a poly(ethylene glycol)-block-poly(propylene glycol)-block-poly (ethylene glycol) (PEO-PPO-PEO) polymer compound;
   a vinyl monomer forming a block copolymer with the PEO-PPO-PEO polymer compound; and
   an active ligand conjugating with the block copolymer.
5. A pharmaceutical composition, comprising:
   a pharmaceutical acceptable carrier and a {PPEO}-{AFG}-{DV} polymer,
   wherein the {PPEO} is a poly (ethylene glycol)-block-poly (propylene glycol)-block-poly (ethylene glycol) (PEO-PPO-PEO) polymer compound, the {AFG} is a vinyl monomer and the {DV} is an active ligand.
6. According to the above embodiments, wherein the PEO-PPO-PEO polymer compound is selected from a group consisting of Pluronic L35, Pluronic L43, Pluronic L44, Pluronic L61, Pluronic L62, Pluronic L64, Pluronic L81, Pluronic L92, Pluronic L101, Pluronic L121, Pluronic P84, Pluronic P85, Pluronic P103, Pluronic P104, Pluronic P105, Pluronic P123, Pluronic F68, Pluronic F87, Pluronic F88, Pluronic F98, Pluronic F108, Pluronic F127 and a combination thereof.
7. According to the above embodiments, wherein the acrylic acid monomer is selected from a group consisting of an acrylate, an acrylamide, a methylacrylamide, a methacrylate and a combination thereof.
8. According to the above embodiments, wherein the vinyl monomer is selected from a group consisting of 2-hydroxyethyl acrylate (HEA), tert-butyl acrylate (tBA), glycidyl acrylate (GA) and a combination thereof.
9. According to the above embodiments, wherein the acrylamide is dimethylacrylamide.
10. According to the above embodiments, wherein the methacrylate is selected from a group consisting of 2-(diethylamino)ethyl methacrylate (DEAEMA), 2-(dimethylamino)ethyl methacrylate (DMAEMA), 2-(diisopropylamino)ethyl methacrylate (DPAEMA), (2-hydroxy-3-(2-aminoethyl)amino)propyl methacrylate (HAEAPMA), glycidyl methacrylate (GMA), poly(ethylene glycol) methacrylate (PEGMA), poly(glycidyl methacrylate) (PGMA) and a combination thereof.
11. According to the above embodiments, wherein the methylacrylamide is selected from a group consisting of methacryloxysuccinimide (MAS), 2-lactobionamidoethyl methacrylamide (LAEMA), N-[3-(dimethylamino)propyl]methacrylamide (DMAPMA), 2-aminoethyl methacrylate (AEMA), 3-aminopropyl methacrylamide (APMA), N-(2-hydroxyethyl)methacrylamide (HEMA), N-(2-hydroxypropyl)methacrylamide (HPMA) and a combination thereof.
12. According to the above embodiments, wherein the active ligand is selected from a group consisting of a folic acid, an arginine-glycine-aspartate (Arg-Gly-Asp, RGD) sequence, a transferrin, an Angiopep, a chlorotoxin and a combination thereof.
13. According to the above embodiments, wherein the Angiopep is selected from a group consisting of Angiopep-1, Angiopep-2, Angiopep-3, Angiopep-4a, Angiopep-4b, Angiopep-5, Angiopep-6, Angiopep-7 and a combination thereof.
14. A method for administering a pharmaceutical nanoparticle, comprising steps of:
   preparing an anti-cancer drug with a {PPEO}-{AFG}-{DV} polymer to form the pharmaceutical nanoparticle, wherein the {PPEO} is a poly(ethylene glycol)-block-poly(propylene glycol)-block-poly(ethylene glycol) (PEO-PPO-PEO) polymer compound, the {AFG} is a vinyl monomer and the {DV} is an active ligand; and administering the pharmaceutical nanoparticle to a subject in need thereof.
15. A method for administering a pharmaceutical micelle, comprising steps of:
   polymerizing an anti-cancer drug with a {PPEO}-{AFG}-{DV} polymer to form the pharmaceutical micelle, wherein the {PPEO} is a poly(ethylene glycol)-block-poly(propylene glycol)-block-poly(ethylene glycol) (PEO-PPO-PEO) polymer compound; the {AFG} is a vinyl monomer and the {DV} is an active ligand, and
   administering the pharmaceutical micelle to a subject in need thereof.
16. A method for administering a pharmaceutical nanoparticle, comprising steps of:
polymerizing a plasmid DNA with a {PPEO}-{AFG}-{DV} polymer to form the pharmaceutical nanoparticle, wherein the {PPEO} is a poly(ethylene glycol)-block-poly(propylene glycol)-block-poly(ethylene glycol) (PEO-PPO-PEO) polymer compound; the {AFG} is a vinyl monomer and the {DV} is an active ligand, and
   administering the pharmaceutical nanoparticle to a subject in need thereof.
17. A method for administering a pharmaceutical micelle, comprising steps of:
   polymerizing a plasmid DNA with a {PPEO}-{AFG}-{DV} polymer to form the pharmaceutical micelle, wherein the {PPEO} is a poly(ethylene glycol)-block-poly(propylene glycol)-block-poly(ethylene glycol) (PEO-PPO-PEO) polymer compound; the {AFG} is a vinyl monomer and the {DV} is an active ligand, and
   administering the pharmaceutical micelle to a subject in need thereof.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

### REFERENCE

1. Layman, J. M. et. al., Influence of polycation molecular weight on poly(2-dimethylaminoethyl methacrylate)-mediated DNA delivery in vitro. Biomacromolecules 2009, 10 (5), 1244-52.
2. Agarwal, A. et. al., Novel cationic pentablock copolymers as non-viral vectors for gene therapy. J Control Release 2005, 103 (1), 245-58.
3. Ting-Fan Yang, Crosslinked Pluronic Micelle as a Carrier for Drug Delivery. 2005.
4. Matyjaszewski, K.; Xia, J., Atom transfer radical polymerization. Chem Rev 2001, 101 (9), 2921-90.
5. Ge, H. et. al., Preparation, characterization, and drug release behaviors of drug nimodipine-loaded poly(epsilon-caprolactone)-poly(ethylene oxide)-poly(epsilon-caprolactone) amphiphilic triblock copolymer micelles. J Pharm Sci 2002, 91 (6), 1463-73.

What is claimed is:

1. A nanoparticle, comprising:
a poly(ethylene glycol)-block-poly(propylene glycol)-block-poly (ethylene glycol) (PEO-PPO-PEO) polymer compound;
a vinyl monomer forming a block copolymer with the PEO-PPO-PEO polymer compound; and
an active ligand conjugating with the block copolymer.

2. A nanoparticle as claimed in claim 1 further comprising a pharmaceutically acceptable carrier.

3. A nanoparticle as claimed in claim 2, wherein the nanoparticle is a pharmaceutical composition.

4. A nanoparticle as claimed in claim 1, wherein the PEO-PPO-PEO polymer compound is selected from a group consisting of Pluronic L35, Pluronic L43, Pluronic L44, Pluronic L61, Pluronic L62, Pluronic L64, Pluronic L81, Pluronic L92, Pluronic L101, Pluronic L121, Pluronic P84, Pluronic P85, Pluronic P103, Pluronic P104, Pluronic P105, Pluronic P123, Pluronic F68, Pluronic F87, Pluronic F88, Pluronic F98, Pluronic F108, Pluronic F127 and a combination thereof.

5. A nanoparticle as claimed in claim 1, wherein the vinyl monomer is selected from a group consisting of an acrylate, an acrylamide, a methylacrylamide, a methacrylate and a combination thereof.

6. A nanoparticle as claimed in claim 5, wherein the acrylate is selected from a group consisting of 2-hydroxyethyl acrylate (HEA), tert-butyl acrylate (tBA), glycidyl acrylate (GA) and a combination thereof.

7. A nanoparticle as claimed in claim 5, wherein the acrylamide is dimethylacrylamide.

8. A nanoparticle as claimed in claim 5, wherein the methacrylate is selected from a group consisting of 2-(diethylamino)ethyl methacrylate (DEAEMA), 2-(dimethylamino)ethyl methacrylate (DMAEMA), 2-(diisopropylamino)ethyl methacrylate (DPAEMA), (2-hydroxy-3-(2-aminoethyl)amino)propyl methacrylate (HAEAPMA), glycidyl methacrylate (GMA), poly(ethylene glycol) methacrylate (PEGMA), poly(glycidyl methacrylate) (PGMA) and a combination thereof.

9. A nanoparticle as claimed in claim 5, wherein the methylacrylamide is selected from a group consisting of methacryloxysuccinimide (MAS), 2-lactobionamidoethyl methacrylamide (LAEMA), N-[3-(dimethylamino)propyl]methacrylamide (DMAPMA), 2-aminoethyl methacrylate (AEMA), 3-aminopropyl methacrylamide (APMA), N-(2-hydroxyethyl)methacrylamide (HEMA), N-(2-hydroxypropyl)methacrylamide (HPMA) and a combination thereof.

10. A nanoparticle as claimed in claim 1, wherein the active ligand is selected from a group consisting of a folic acid, an arginine-glycine-aspartate (Arg-Gly-Asp, RGD) sequence, a transferrin, an Angiopep, a chlorotoxin and a combination thereof.

11. A nanoparticle as claimed in claim 10, wherein the Angiopep is selected from a group consisting of Angiopep-1, Angiopep-2, Angiopep-3, Angiopep-4a, Angiopep-4b, Angiopep-5, Angiopep-6, Angiopep-7 and a combination thereof.

12. A nanoparticle, comprising:
a {PPEO}-{AFG}-{DV} polymer, wherein the {PPEO} is a poly (ethylene glycol)-block-poly (propylene glycol)-block-poly (ethylene glycol) (PEO-PPO-PEO) polymer compound; the {AFG} is a vinyl monomer and the {DV} is an active ligand.

13. A method for administering a pharmaceutical nanoparticle, comprising steps of:
polymerizing an effective amount of a pharmaceutical compound with a {PPEO}-{AFG}-{DV} polymer to form the pharmaceutical nanoparticle, wherein the {PPEO} is a poly(ethylene glycol)-block-poly(propylene glycol)-block-poly(ethylene glycol) (PEO-PPO-PEO) polymer compound; the {AFG} is a vinyl monomer and the {DV} is an active ligand, and
administering the pharmaceutical nanoparticle to a subject in need thereof.

14. A method as claimed in claim 13, wherein the pharmaceutical compound is selected from a group consisting of a nonsteroidal anti-inflammatory drug, a steroid, an anticancer drug, a plasmid DNA and a combination thereof.
